(19) United States
(12) Patent Application Publication
(10) Pub. No.: US 2020/0254110 A1
(43) Pub. Date: Aug. 13, 2020

(54) LIGAND-CONJUGATES AND METHODS FOR TARGETED RECEPTOR-MEDIATED CELLULAR UPTAKE

(71) Applicant: UNIVERSITY OF BREMEN, Bremen (DE)

(72) Inventors: Ursula MIRASTSCHIJSKI, Bremen (DE); Mario WAESPY, Bremen (DE); Sørge KELM, Lilienthal (DE)

(21) Appl. No.: 16/650,262

(22) PCT Filed: Sep. 25, 2018
(86) PCT No.: PCT/EP2018/075955

§ 371 Date: Mar. 24, 2020

(30) Foreign Application Priority Data
Sep. 25, 2017 (EP) 17192916.9

(51) Int. Cl. A61K 47/66; A61K 47/60; A61K 47/54; A61K 47/69

(52) U.S. Cl. CPC A61K 47/66; A61K 47/60; C07K 7/06; A61K 47/6929; A61K 47/541

(57) ABSTRACT

Provided are ligand-conjugates and methods for targeted receptor-mediated cellular uptake of an entity of interest of desired activity and function.

### FIELD OF THE INVENTION

The present invention relates to novel protease-activated receptor (PAR) ligand-conjugates for receptor-mediated signal transduction and cellular uptake of entities of desired activity and function.

### BACKGROUND TO THE INVENTION

A number of methods are known for selectively targeting cells in a patient for delivery of diagnostic or therapeutic agents. Selective targeting has led to the introduction of various entities of interest including diagnostic agents for visualization of tissues, such as contrast agents useful in Magnetic Resonance Imaging (MRI), radio-diagnostic compositions, and the like. Introduction of therapeutic agents, such as compositions for radiotherapy or for neutron capture therapy, compositions for chemotherapy, various proteins, peptides, and nucleic acids, protein toxins, anti sense oligonucleotides, liposomes, analgesics, antibiotics, antihypertensive agents, antiviral agents, antihistamines, expectorants, vitamins, plasmids, and the like, has also been demonstrated. For example, folate conjugates have been used for the selective targeting of cell populations expressing folate receptors or other folate binding proteins to label or deliver bioactive compounds to such cells. Efforts to improve the selectivity of targeting or increase the diversity of the agents delivered to the cell or tissue by receptor ligand-conjugates have been hampered by a number of complications, including suitable alternative receptors on target cells or tissue and the complex syntheses required for the preparation of these conjugates.

Thus, the technical problem underlying the present invention was to provide ligand-conjugates and methods for targeted receptor-mediated cellular uptake of an entity of interest such as mentioned above. The solution to the technical problem is achieved by providing the embodiments characterized in the claims and described further below.

### SUMMARY OF THE INVENTION

In a general aspect, the present invention relates to novel receptor ligand-conjugates suitable for targeted receptor-mediated signal transduction and cellular uptake of entities of desired activity and function. The present invention is based on the synthesis of a novel class of PAR (protease activated receptor) ligands illustrated in the Examples with a synthetic PAR2 agonist denoted P8, which are able to specifically bind and activate cell surface PAR, in a manner similar to the native activation process mediated by several serine proteases. Activation of the receptor initiates a number of intracellular signal cascades and downstream signaling events, as well as the β-arrestin-mediated and clathrin- and dynamin-dependent endocytosis of PAR itself. In this regard, the novel class of PAR ligands such as the synthetic PAR2 agonist P8 can be employed to trigger endocytosis and enables the target-specific uptake of molecules, proteins and other biofunctional material or particles into cells, when directly linked to them. Hence, the ligand-conjugates of the present invention pave the way for a new generation of pharmacological, therapeutical, diagnostic and biotechnological applications, such as target drug delivery or directed cell engineering.

The disclosure of the present invention includes (1) the design of the PAR ligand and ligand-conjugate, (2) synthesis of a novel class of PAR ligands, (3) PAR-ligand interaction on the atomic level (ligand docking simulations) assisting in the design of novel PAR ligands and PAR ligand-conjugates, (4) cellular assays for proof-of-concept such as verifying the biological function of the PAR ligand in PAR-mediated cell activation, and (5) biological function of the ligand and ligand-conjugate, respectively, enhancing PAR-mediated uptake by eukaryotic cells. In order to achieve this goal, the present invention provides the synthesis of the novel PAR2 agonist P8 and several assays and methods to test the biofunctionality and biocompatibility, as well as the applicability of the P8 conjugate for potent biochemical and cell biological applications. The following table shortly summarizes the methods employed, the results obtained and possible applications.

TABLE 1
Overview of invention and correlated potential applications.

| | Step | Result | Potential for Applications |
|---|---|---|---|
| 1. | Design of PAR | PAR ligands such as AR2 | A variety of combinations of |
| | ligand, e.g. novel | activating peptide SLIGRL N- | linker, spacer and PAR ligands |
| | PAR2 agonist P8 | terminally conjugated to a | can be realized including use |
| | | spacer and linker molecule for | as a PAR agonist and vehicle |
| | | further derivatization | for PAR ligand-conjugate |
| 2. | Synthesis of novel | Organic synthesis-strategy | A new generation of PAR |
| | PAR ligands such as | development and optimization | ligands, in particular PAR |
| | PAR2 agonist P8 | Purification and | activating agonists, which |
| | | characterization of isolated | allow direct cell controlling, |
| | | PAR ligand such as P8 | reprogramming and |
| | | (HPLC-MS, photometry) | modification through direct |
| | | | receptor-mediated cell |
| | | | activation or specific drug |
| | | | delivery and uptake in a |
| | | | variety of medicinal |
| | | | applications |
| 3. | In situ receptor- | Homology modelling of human | Design of a library of potential |
| | ligand interactions | PAR, for example PAR2 | PAR ligands such as PAR |
| | | Ligand docking simulations | agonists with enhanced |
| | | employing the homology | specificity for directed cell |
| | | model of human PAR2 and P6 | function regulation |
| | | and P8 as model ligands | |
| | | (YASARA) | |
| 4. | Ligand functionality | Cell activation and | Controlling PAR ligand |
| | and cell activation | downstream signaling | functionality provides an |
| | potential of PAR | employing intracellular calcium | essential biotechnical tool for |
| | ligand-conjugates | mobilization assay (Flow | several related medical and |
| | such as P6 and P8 | cytometry) | pharmaceutical applications |
| 5. | Conjugation of PAR | Chemical derivatization of | PAR ligands such as P8 and |
| | ligand such as P8 to | particle surface with PAR | derivatives can be conjugated |
| | selected model | ligand | to a variety of different |
| | particles and uptake | Uptake of the PAR ligand- | molecules, pharmaceutical |
| | into cells | conjugated particles into PAR | compounds, particles or |
| | | expressing cells | microvesicles |
| | | | Model particles can be co- |
| | | | derivatized with PAR ligand |
| | | | such as P8 and other cell type |
| | | | specific receptor ligands e.g. |
| | | | for target specific drug |
| | | | delivery |

The data described in the appended Examples clearly demonstrate the proof of principle of the concept of the present invention for novel endocytosis-triggering ligands. Besides the possibility of generating different PAR ligand derivatives, according to the present invention it will be applicable to other PAR-expressing cell types such as astrocytes and neurons and many other cells, for efficient PAR ligand mediated endocytosis and consequently also on primary cells. While the present invention is illustrated and discussed with human PAR as a preferred embodiment, the person skilled in the art will acknowledge that because PARs are found and conserved in vertebrates and mammalians, the present invention and embodiments are generally applicable, for example to primates, rodents, horses, cows, dogs, cats, fish, pigs, poultry, and the like.

### BRIEF DESCRIPTION OF THE FIGURES

FIG. 1: Design of a PAR ligand in accordance with the present invention illustrated by novel PAR2 agonist P8. A: Energy minimized structure model of the PAR2 agonist P8. B: Lewis structure of the PAR2 agonist P8. 5-FAM: 5-carboxyfluorescein. SLIGRL-NH₂: single letter amino acid code (SerLeuIleGlyArgLeu). The carboxylate at the C-terminus is modified to a primary amide.

FIG. 2: Schematic presentation of the synthesis strategy employed for the generation of a PAR ligand in accordance with the present invention illustrated by the preparation of the novel PAR2 agonist P8.

FIG. 3: Ligand docking simulation of a PAR ligand in accordance with the present invention illustrated by novel PAR2 agonist P8 and the intermediate P6 onto human PAR2. A: Quaternary structure of human PAR2 in complex with novel PAR2 agonist intermediate P6. The molecular surface is shown in blue spheres. B: Human PAR2 in complex with novel PAR2 agonist P8. Carbon atoms are illustrated with green spheres, oxygen with red spheres and nitrogen with blue spheres. Homology model of human PAR2 as well as docking calculations were performed with the software YASARA employing the incorporated Yasara Structure module.

FIG. 4: Downstream calcium-flux determination as a consequence of PAR ligand in accordance with the present invention mediated receptor activation illustrated by PAR2 agonist P8. HEK293 cells were preincubated with the calcium sensitive fluorophore Indo-1 AM and treated with either trypsin, SLIGRL-NH₂ or the synthetic compounds P6 and P8 right before flow cytometric analysis. Black arrows indicate the shift in RFI upon PAR2 activation and subsequent downstream calcium mobilization. SLIGRL-NH₂: single letter amino acid code (SerLeuIleGlyArgLeu-NH₂). DEG-SLIGRL-NH₂: diethylenglycol derivatized SLIGRL-NH₂ (P6). P8: novel PAR2 agonist P8. RFI: relative fluorescence intensity.

FIG. 5: Conjugation of a PAR-ligand in accordance with the present invention to an entity of interest illustrated by PAR2 agonist P8 to iron oxide nanoparticles (IONP) and endocytosis into keratinocytes. DMSA: 2,3-dimercaptosuccinic acid. SLIGRL-NH₂: single letter amino acid code (SerLeuIleGlyArgLeu-NH₂). P8: novel PAR2 agonist P8. RFI: relative fluorescence intensity. Ext. labelling: extended labelling of P8-derivatised IONPs. DAPI: nuclei staining.

FIG. 6: Homology model of human PAR2. A: Comparison of the crystal structure of human PAR1 (PDB: 3VW7) and the homology model of human PAR2. B: Structural alignment of human PAR1 (purple) and PAR2 (light blue). C: Amino acid alignment of human PAR1 and PAR2 sequences. The homology model of PAR2 was generated using the software YASARA and the incorporated YASARA Structure module. The amino acid alignment was generated using the software Geneious.

### DETAILED DESCRIPTION OF THE INVENTION

The present invention generally relates to novel protease-activated receptor (PAR) ligand-conjugates for receptor-mediated signal transduction and cellular uptake of agents, particles, cells and other bioactive molecules. Thus, in its broadest aspect the present invention relates to a conjugate capable of triggering target specific cellular uptake of an entity of interest comprising:

(a) a protease activated receptor (PAR)-binding ligand; and covalently attached thereto

(b) a linker molecule which provides the ability to be chemically linked to the entity of interest; and optionally

(c) the entity of interest conjugated to the linker.

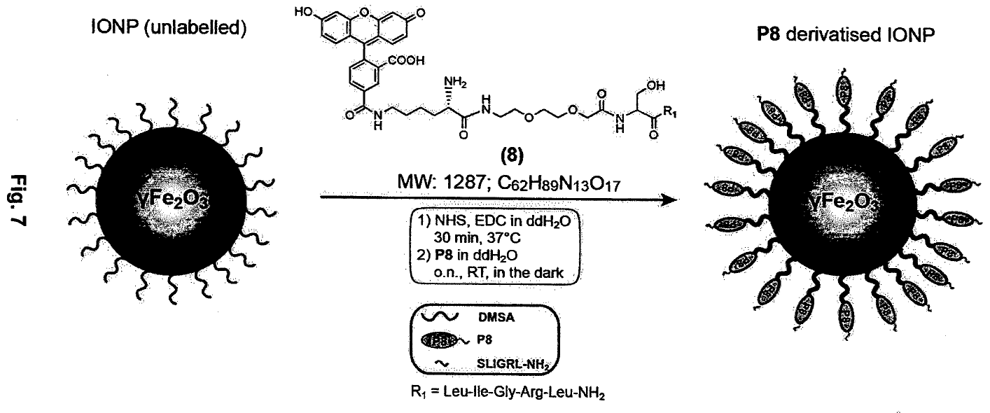

Human protease-activated receptors (PARs), a superfamily of unusual G-protein (GTP binding protein)-coupled receptors (GPCRs) comprising of four family members termed PAR1 through 4 (nomenclature follows [12]) were discovered in the early 90s [13,14] when actually searching for the receptor responsible for platelet agglutination. These type-III transmembrane (TM) receptors share relatively high sequence similarity to other GPCRs such as bovine rhodopsin (PDB 1U19, [15]) and human nociceptin/orphanin FQ/ORL-1 receptor (PDB 4EA3, [16]) regarding their TM region, consisting of seven canonical α-helices (40-50% sequence similarity) [5]. Beside their well-conserved TM region all PARs consist of an extracellular N-and intracellular C-terminus, as well as three extracellular and intracellular loops connecting the TM-segments, respectively (FIG. 6A).

The term “unusual”, used above when classifying PARs to GPCRs, refers to the process of receptor-activation. PARs are predominantly activated by proteases via proteolytic cleavage of their extracellular N-terminus e.g. at Ser-37 in case of PAR2, leading to the exposure of a new amino terminus with the sequence Ser-Leu-Ile-Gly-Arg-Leu (SLIGRL). This newly formed N-terminus serves as a tethered ligand (TL, FIG. 6C) and is in turn able to fold back and intra-molecularly bind to the extracellular part of the receptor, consequently initiating receptor activation and subsequent signal transduction [17-22]. The receptor remains inactive as long as this TL sequence is “protected” by the 36 amino acid long N-terminus. So far, a number of PAR-activating proteases have been identified, including proteases, e.g. from inflammatory cells, the digestive tract or the coagulation cascade, such as thrombin [14], trypsin [18], tryptase [23], factor VIIa and Xa [24,25], granzyme A [26], matriptase [27], kallikreins (KLK2, 4, 5, 6, 14) [28-30] and other serine proteases but also the matrix metalloproteinase-1 (MMP-1) in case of PAR1 activation [31]. Hence, apart from their traditional function as classical protein digesting enzymes, proteases become more recognized as hormone-like, cell regulatory molecules involved in a number of pathological and physiological processes.

The general mechanisms of PAR cleavage and activation have been intensively investigated over the past decades [17,22,32-35]. Interestingly, in contrast to PAR1, 3 and 4, PAR2 is the only member of the superfamily, which cannot be activated through thrombin but is predominately cleaved and activated by pancreatic trypsin [18,36-38]. The reason for this observation is a negatively charged hirudin-like region that interacts with the exosite of thrombin, which was found in PAR1 and 3 but is missing in case of PAR2. PAR4 was found to directly interact with the active center of thrombin [34]. PAR activation involves a number of G-proteins and initiates a wide range of different signal transduction pathways in a number of cell types with diverse consequences and events that mediates processes like inflammation, pain, homeostasis and repair mechanisms [21,39]. For example it has been shown that PAR2 couples to several G-protein α-subunits, such as Gαᵢ, Gα_{q}, Gαₛ and Gα₁₂₋₁₃ [40]. Following PAR activation, intracellular signal pathways are triggered via extracellular signal-regulated kinase-1 and 2 (ERK1/2) phosphorylation [2,40,41], RhoA GTPase activation [42], adenylyl cyclase inhibition [43] and mitogen-activated protein kinase (MAPK) pathway activation [44], as well as production of inositol triphosphate (IP3) and diacyl glycerol (DAG) and downstream mobilization of intracellular Ca²⁺ [45] finally resulting in enhanced transcription, mitogenesis, cell growth and differentiation. Furthermore, activated PAR2 can also be phosphorylated via G-protein-coupled receptor kinases (GRKs) [46,47] leading to β-arrestin 1 and 2 recruitment [22,48], which in turn uncouples PAR2 from G-protein complexes consequently terminating PAR2 induced signaling and mediate dynamin- and clathrin-dependent endocytosis of PAR2 [49,50].

In summary, the diverse involvement of PARs in different cellular processes is due to their frequent appearance in various cell types, although PAR2 represents the most widespread receptor of the PAR family [61] and therefore obtains major attention in the research field. Besides PAR-activating proteases, a number of proteases are also capable of preventing PAR mediated cell activation and downstream signaling upon proteolytic cleavage. The underlying mechanisms of this negative PAR regulation comprise either the cleavage of the extracellular amino terminus C-terminally distant from the TL sequence, thus removing the same and consequently disarming the receptor or by cleaving elsewhere in the latter to disable signaling [62,63]. One example for such a PAR-disarming protease represents Cathepsin G, a serine protease expressed by activated neutrophils at sites of injury and inflammation [64]. Molino et al. demonstrated that Cathepsin G treatment of PAR expressing cell lines abolished thrombin induced cell activation, indicating the modification or clearance of the thrombin cleavage site as a consequence of the proteolytic activity of Cathepsin G [65].

Furthermore, it has been suggested that all PARs are exclusively activated via proteolytic cleavage of their extracellular N-terminus in vivo, since no endogenous PAR-ligand molecules have been identified so far. Besides that, it has been proposed that PAR3 rather directly mediates cell signaling through self-activation but more function as a cofactor for PAR4 activation [66]. Four years later, Hansen et al. demonstrated that synthetic peptides derived from the TL sequence of PAR3 were indeed able to activate PAR2 besides PAR1 [67], indicating the complexity in the regulation of PAR-mediated cell signaling.

In accordance with the present invention, the mechanism of receptor silencing and endocytosis after activation is utilized to prevent permanent cell stimulation and represents the strategy for the internalization of PAR ligand-conjugates, illustrated in the Examples with PAR2 agonist P8 labelled nanoparticles; see Examples 4 and 5. Hence, in a further preferred embodiment of the conjugate of the present invention, the PAR to be targeted is PAR2, preferably human PAR and PAR2, respectively.

As mentioned, it has been suggested that all PARs are exclusively activated via proteolytic cleavage of their extracellular N-terminus consequently exposing the PAR activating tethered ligand (TL) sequence in vivo, since no endogenous PAR-ligand molecules have been identified so far. However, it has been demonstrated that synthetic peptides, such as SLIGRL-NH2 (native TL of rodent PAR2) and SLIGKV-NH2 (native TL of human PAR2), derived from the TL sequence of PARs were indeed able to mimic the native ligand and activate PAR2 and PAR1 [67]. After these ground-breaking observations several research groups started the design and synthesis of a small library of new PAR-activating ligands, including native TL mimicking peptides as well as full synthetic PAR-agonist compounds [1-3,35,67-76]. Thus, PAR ligands, in particular PAR activating peptides which may be used and adapted in accordance with the present invention are well known in the art; see also, e.g., Zhao et al., Frontiers in Endocrinology 5 (2014), 1-16, especially Tables 1 to 3 and the appended Examples. A recent review [75] on patents regarding PAR modulating peptides is attached to this description. Hoffmann et al., Bioconjugate Chem. 23 (2012), 2098-2104 describes a conjugate comprising a PAR2-binding ligand (2-f-LIGRL) covalently attached to a dtpa via an ornithine linker unit, which is taught to be used as a chelator for Eu²⁺. U.S. patent application 2006/0104944 A1 describes conjugates of PAR2-binding ligands, including SLIGRL-NH₂ and SLIGKV-NH₂ with polymers such as PEG, and also conjugates of PAR2 agonist peptides with antibodies. Flynn et al., FASEB J. 27 (2013), 1498-1510 describes a derivative of SLIGRL bound to a palmitoyl group (PAM) via polyethylene glycol linkers. None of the documents discloses or suggests that such conjugates are capable of triggering specific cellular uptake of an entity of interest.

In a preferred embodiment of the conjugate of the present invention, the ligand is a peptide, preferably PAR activating peptide or derivative thereof including but not limited to peptoids, PNAs, biomimetics and the like, preferably wherein the peptide has a serine at the N-terminus.

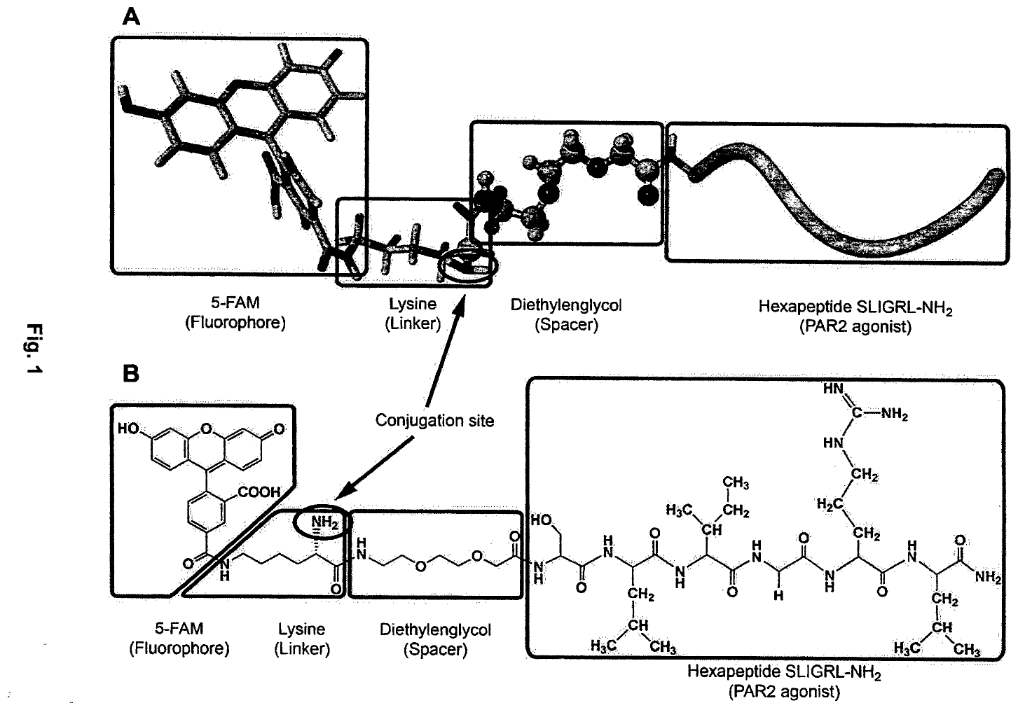

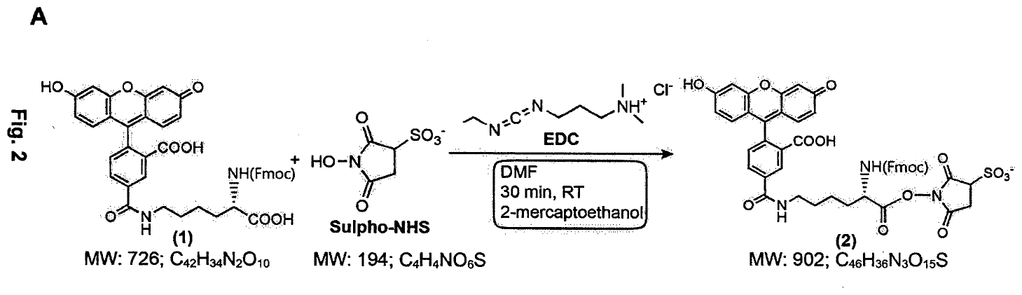

In addition or alternatively, the carboxylate at the C-terminus of the peptide is modified so as to reduce to the reactivity of the peptide at its C-terminus; see FIG. 1. Preferably, the carboxylate at the C-terminus of the peptide is modified to a primary amide. In a particular preferred embodiment of the conjugate of the present invention, the PAR ligand, i.e. PAR activating peptide comprises or consist of the amino acid sequence SLIGRL (SEQ ID NO: 1), SLIGKV (SEQ ID NO: 3), or a derivative thereof, preferably SLIGRL-NH₂ or SLIGKV-NH₂.

In a further preferred embodiment of the conjugate of the present invention the linker is directly or indirectly attached to the N-terminus of the peptide, preferably at the free amine of the serine. However, as explained in the Examples, mutation of the serine to threonine retained ligand activity, suggesting that the correct positioning of a polar group,

such as sulphydryl or hydroxyl group of the amino acid side chain, as in serine or threonine is important for ligand binding. Therefore, other amino acids such as threonine or functional groups may be present at the N-terminus of the PAR ligand for attaching the linker and spacer, respectively.

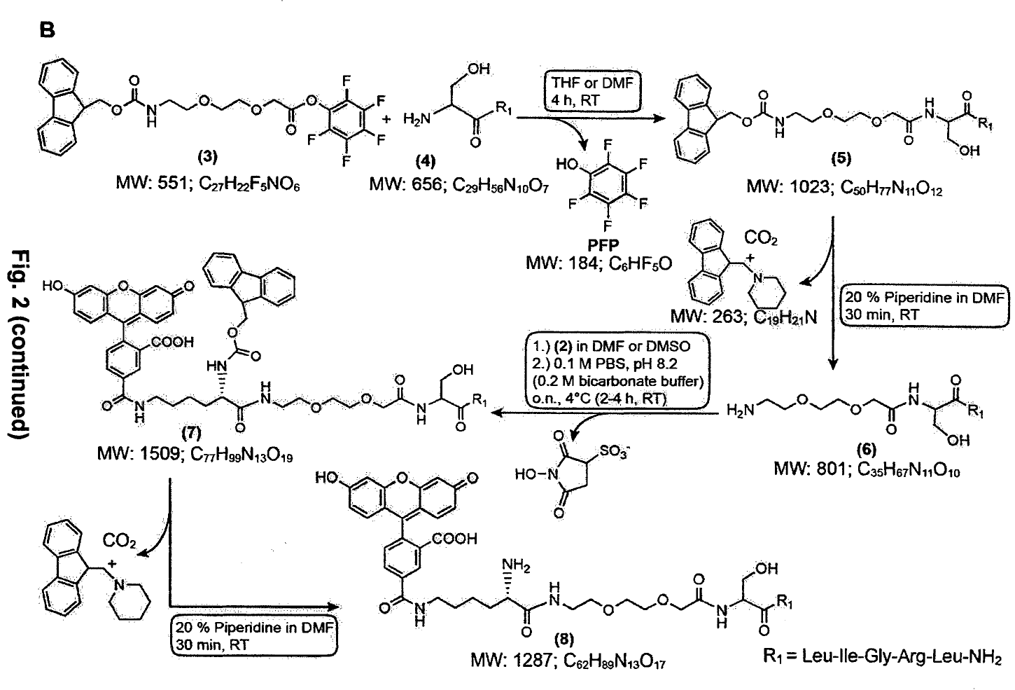

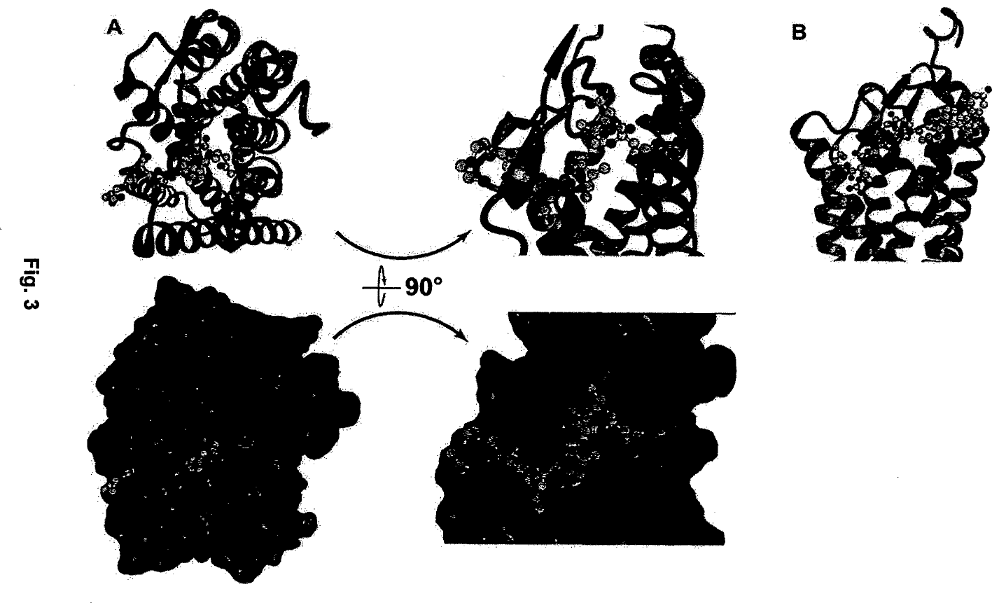

As illustrated in FIGS. 1 and 2, and common in the preparation of receptor binding ligands, in particular peptides for target-selective delivery of an entity of interest, linkers such as amino acids comprise an amino or thiol group for conjugation and preserve the affinity for the receptor are known in the art; see, e.g., for review Chen et al. Adv. Drug Deliv. Rev. 65 (2013), 1357-1369 and Accardo et al., Int. J. Nanomedicine 9 (2014), 1537-1557. In a preferred embodiment of the conjugate of the present invention the linker molecule is lysine.

In a further preferred embodiment of the conjugate of the present invention the linker is attached to the ligand by a spacer molecule; see FIGS. 1 and 2. Like linkers, spacer molecules such as those consisting of ethylenglycol units are well known in the field; see, e.g., for review Hamley, Biomacromolecules 15 (2014), 1543-1559 as well as Chen (2013) and Accardo (2014), supra. Preferably, the spacer molecule is diethylenglycol (DEG). However, it should be noted, that based on the molecular modelling data provided in Example 3 the spacer can be modified in length and structure to modulate the interactions between PAR and the ligand. In particular, this includes strengthening the binding by interactions outside of the binding site for the activating peptide. For example, while two ethylenglycol units (DEG) have been used in the Examples, it may well be extended to oligo- and polyethylenglycol (PEG) depending on specific demands of an application. For example, when the PAR-ligand, e.g. PAR2 activating peptide will be attached to complex surfaces of micro vesicles or other nano-/micro-particles, it might be critical that the PAR2 activator peptide is more exposed to the environment and distant from the carrier surface.

In a further preferred embodiment of the conjugate of the present invention the linker comprises a detectable label, preferably a fluorophore such as 6-carboxyfluorescein (6-FAM) and 5-carboxyfluorescein (5-FAM) or a mixture thereof. One significant advantage in relation to this embodiment includes the possibility to monitor the time dependent internalization of the PAR ligand and PAR ligand-conjugate into cells. In case of a fluorophore 6-FAM and 5-FAM monitoring of the PAR ligand labelled entity of interest into cells can be performed without any chemical interference of the label, which will be discussed more in detail below.

As mentioned the ultimate goal of the present invention is to provide ligand-conjugates and methods for targeted receptor-mediated cellular uptake of an entity of interest such as mentioned above in vitro or in vivo, i.e. within a subject. An “entity of interest” includes any therapeutic or diagnostic functional entity selected from but not limit to any medical, diagnostic, pharmaceutical or biological entity whose delivery to a targeted site in a subject has therapeutic benefit and/or diagnostic value and which entity can be linked to a the PAR ligand of the present invention. Examples of therapeutic or diagnostic functional entities include a nucleic acid; a protein; a peptide; a gene delivery vehicle (such as a plasmid, a virus, a liposome complex); an enzyme; a thrombolytic agent; an anticoagulant; a chemotherapeutic agent; an apoptotic agent; a pharmaceutical; a chemical compound; a growth factor; a cytokine; other ligands for cell surface receptors; a carbohydrate, a lipid, imaging agents, such as radiochemicals, fluorescence chemicals, metal ions that can be detected externally; or a cosmetic agent such as for regulating the humidity of the skin, skin tanning, blood circulation of the skin and the like. For suitable agents see also international applications WO 2002/085908 and WO 2003/009815 as well as Accardo et al. (2014), supra. In one embodiment of the conjugate of the present invention the entity of interest is a diagnostic or therapeutic agent, a cell, a micro-vesicle or other nano- or micro-particle, preferably iron oxide nanoparticle (IONP); see also Example 5.

As mentioned above and illustrated in the Examples, in the course of preparing the PAR ligand-conjugates of the present invention also novel PAR ligands such as PAR2 agonist P6 and P8 have been provided on the basis of PAR activating peptides which are modified at their N-terminus, for example with a functional moiety such as a spacer or linker, preferably at a serine at the N-terminus; see supra. In addition or alternatively, the carboxylate at the C-terminus of the peptide is modified so as to reduce to the reactivity of the peptide at its C-terminus; see FIG. 1. Preferably, the carboxylate at the C-terminus of the peptide is modified to a primary amide. In a particular preferred embodiment of the modified PAR activating peptide comprises or consist of the amino acid sequence SLIGRL (SEQ ID NO: 1), SLIGKV (SEQ ID NO: 3), or a derivative thereof, preferably SLIGRL-NH2 or SLIGKV-NH2. Accordingly, in a further aspect the present invention relates a PAR ligand comprising or consisting of a PAR activating peptide or derivative thereof such as defined herein before and illustrated in the Examples, which is modified with a functional moiety at its N-terminus, preferably at a serine, preferably wherein the functional moiety is spacer or linker molecule.

It will be appreciated by the person skilled in the art that the components of the compositions of the present invention are typically provided in the form of one or more kits or pharmaceutical and diagnostic compositions, respectively, each containing an effective amount of the conjugate and novel PAR ligand of the present invention together with a pharmaceutically-acceptable buffer, excipient, diluent or carrier as well as instruction for use and/or optionally suitable means for detection of the ligand. Accordingly, in a further embodiment the present invention relates to a composition and kit comprising the conjugate or novel PAR ligand of the present invention, preferably which is a pharmaceutical or a diagnostic composition, preferably wherein the conjugate comprises the entity of interest. A pharmaceutical composition may include a pharmaceutically acceptable antioxidant. These compositions may also contain adjuvants such as preservatives, wetting agents, emulsifying agents and dispersing agents. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin. Pharmaceutical compositions typically must be sterile and stable under the conditions of manufacture and storage.

Some of the processes in which PARs are proposed to play a crucial role comprise endothelial cell, neuron and astrocyte function [51-54], platelet activation [52], skin pigmentation [55], hyperalgesia (increased perception of pain) and analgesia (absence of sensibility to pain) [56], tumor cell growth and metastasis [57,58], as well as HIV-induced neuroinflammation [51]. For example, Noorbakhsh and co-workers detected increased mRNA levels of PAR1 and prothrombin in brain samples form patients infected with HIV (and suffering AIDS) relative to controls [51]. Another interesting finding was made by D'Andrea et al. who observed the upregulation of PAR1 and PAR2 in human stromal fibroblasts surrounding the malignant breast cancer cells, besides the presence of PAR1 and PAR2 mRNA and protein in the latter [59]. It should be noted here that besides the significant increasing incidences of breast cancer, over the past decades, in a number of developing countries also the high disease-related mortality, due to inefficient therapies remains a present challenge. Thus the identification of new therapeutic targets as well as the development of new strategies to more efficiently fight cancer represents an essential issue. Accordingly, in one aspect the present invention relates to the conjugate of the present invention disclosed hereinbefore and illustrated in the Examples and compositions described herein for use in the treatment, diagnosis or monitoring of a disease or condition related to the over-expression of PAR in a cell, preferably wherein the disease is cancer. In another embodiment, conjugate or composition of the present invention is used for the modulation of a cell expressing PAR in vitro. In addition, Freund-Michel and co-workers proposed the role of PAR2 in inflammatory airways disease, such as asthma. They observed the upregulation of PAR2 in primary cultures of human airway smooth muscle cells (HASMC) under inflammatory conditions (treatment with cytokine IL-1β) [60]. Therefore, it is prudent to expect that the conjugate and novel PAR ligand of the present invention are useful in the treatment or investigation of those diseases as well.

As mentioned hereinbefore, the disclosure of the present invention includes the design of the ligand and ligand-conjugate, synthesis of the novel class of PAR ligands, and PAR-ligand interaction on the atomic level (ligand docking simulations) assisting in the design of novel ligands and ligand-conjugates; see also Table 1, supra, and the Examples. Accordingly, in a further aspect the present invention relates to a method of preparing a PAR agonist or a conjugate capable of triggering target specific cellular uptake of an entity of interest comprising:

(a) coupling a spacer molecule (3) at the free amine group of the N-terminal serine of a PAR ligand, preferably a PAR activating peptide (4) so as to obtain an intermediate modified PAR ligand (5, 6), preferably a PAR agonist (6); and optionally

(b) coupling a bi-or multivalent linker (1, 2) which provides the ability to be chemically linked to the entity of interest to the spacer molecule of the modified PAR ligand (6), preferably wherein the linker comprises a detectable label (7), so as to obtain a further modified PAR ligand (8); and optionally

(c) conjugating the entity of interest to the linker, preferably via a free amino group of the linker so as to obtain a conjugate of the PAR ligand and the entity of interest;

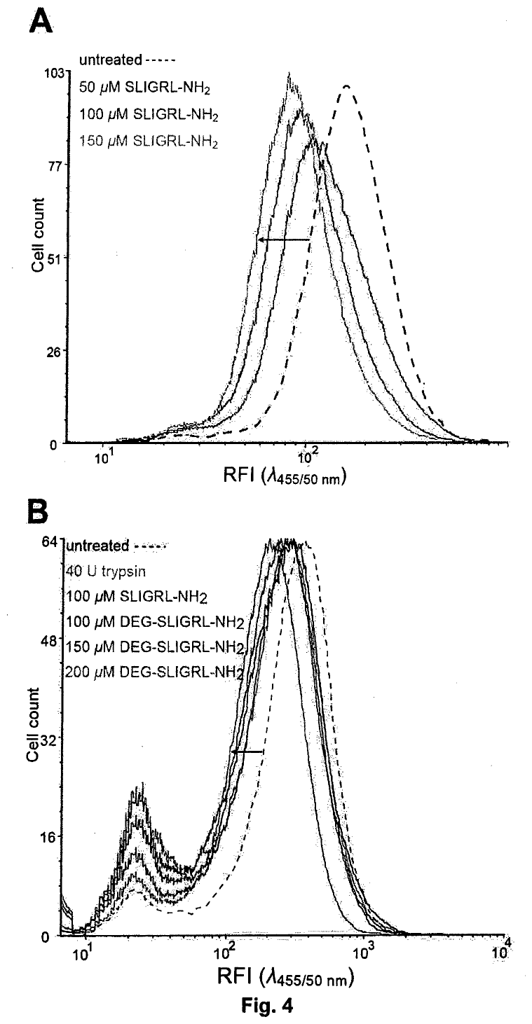

see also Examples 1 and 2 as well as FIGS. 1 and 2. Preferably, prior, during or after any one of steps (a) to (c) the potential intermolecular receptor-ligand interaction is determined based on ligand-docking simulations using a homology model of PAR, preferably human PAR2 together with the intermediate ligand or conjugate either with or without the entity of interest; see Example 3 and FIGS. 3 and 6. Since the present invention also provides means and methods for preparing the PAR ligand-conjugates disclosed herein, the present invention also relates to the use of a PAR ligand, linker, spacer, detectable label, or entity of interest to be delivered into a target cell for the preparation of the conjugate and the novel PAR agonist or the composition comprising the same.

The above disclosure generally describes the present invention. Unless otherwise stated, a term as used herein is given the definition as provided in the Oxford Dictionary of Biochemistry and Molecular Biology, Oxford University Press, 1997, revised 2000 and reprinted 2003, ISBN 0 19 850673 2. Several documents are cited throughout the text of this specification, either by direct reference or numbering in parenthesis and listed separately. The contents of all cited references (including literature references, issued patents, published patent applications as cited throughout this application including the background section and manufacturer's specifications, instructions, etc.) are hereby expressly incorporated by reference; however, there is no admission that any document cited is indeed prior art as to the present invention. A more complete understanding can be obtained by reference to the following specific Examples which are provided herein for purposes of illustration only and are not intended to limit the scope of the invention.

### EXAMPLES

### Example 1: Design of Novel PAR Ligands

The innovative aspect of the invention includes the regio-selective conjugation of a PAR ligand illustrated by a PAR2-activating peptide with a suitable linker molecule, which provides the ability to chemically link the PAR2 agonist P8 to a certain target molecule, while maintaining its capability of receptor activation (FIG. 1). In brief, diethylenglycol (DEG) has been used as a spacer molecule bridging the hexapeptide SLIGRL-NH₂ (PAR2 agonist, SerLeuIleGlyArgLeu-NH₂) with the linker molecule lysine (FIG. 1 A,B). This DEG spacer, besides its high conformational flexibility, provides a defined distance between the PAR-ligand, here PAR2-activating peptide and the amine at the alpha-carbon of lysine, which is required for further conjugation of the novel PAR2 agonist P8 to selected target molecules. It should be noted, that based on the molecular modelling data provided in Example 3 the spacer can be modified in length and structure to modulate the interactions between PAR and the ligand. In particular this includes strengthening the binding by interactions outside of the binding site for the activating peptide. For example, while here two ethylenglycol units (DEG) have been used, it may well be extended to polyethylenglycol (PEG) depending on specific demands of an application. For example, when the PAR-ligand, e.g. PAR2 agonist will be attached to complex surfaces of micro vesicles or other nano-/micro-particles, it might be critical that the PAR ligand is more exposed to the environment and distant from the carrier surface.

Finally, the amino acid side chain of the linker, here lysine is derivatized with a detectable label, here 5-carboxyfluorescein (5-FAM, FIG. 1), a common fluorophore, which enables photochemical detection, as well as quantification of the novel PAR ligand after its attachment to selected target molecules. Together with a cell type specific receptor ligand, such PAR ligand labelled particles can be employed as target specific drug delivery systems with enhanced therapeutic efficiency at target cells or tissues. One significant advantage in relation to this includes the possibility to monitor the time dependent internalization of PAR ligand labelled particles, e.g. PAR2 agonist P8-labelled particles into cells without any chemical interference of the 5-FAM, which will be discussed more in detail below.

Interestingly, a recent publication focusing on PAR2-selective agonist mutagenesis demonstrated that the first two residues 2-furoyl and leucine in the potent PAR2 agonist peptide 2-furoyl₍₁₎L₍₂₎IG₍₄₎RL₍₆₎-NH₂, a 10-20 fold more effective derivative of SLIGRL-NH₂ [1], are the crucial factors for ligand affinity and activity relative to the glycine and leucine at position 4 and 6 in the peptide [2]. In summary, it can be specified that the N-terminal part of the PAR2 agonist peptide depict the more crucial region for ligand binding, specificity and PAR activation. In contrast to the current knowledge publicly available, the novel approach of the present invention, using DEG as a relatively linear and sterically simple linker for the derivatization of the serine in SLIGRL-NH₂, surprisingly and against all expectations lead to an active compound exhibiting PAR2 activation and downstream signaling in vitro and therefore represents a complete new type of synthetic PAR agonists.

### Example 2: Synthesis of Novel PAR Ligands

In order to isolate and biochemically characterize the novel PAR ligands a synthesis strategy (FIG. 2) has been established and optimized by way of illustration with the novel PAR2 agonist P8. However, though less preferred other synthesis strategies could be used to obtain ligands of this invention.

Formation of Fam5/6-Lysine(Fmoc)-NHS-ester P2

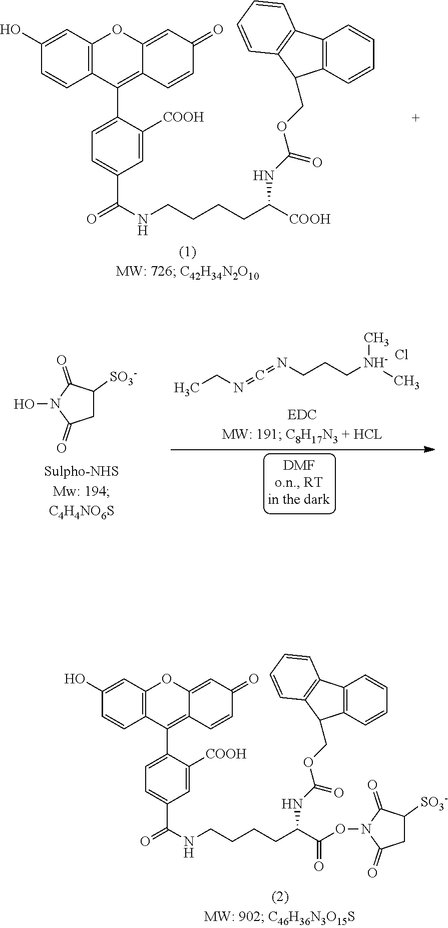

200 mg (275.1 μmol, 1 eq.) of Fam5/6-Lysine(Fmoc) (1) were dissolved in 30 mL of fresh and amine free DMF and transferred into a 100 mL reaction flask. 262.72 mg (1.375 mmol, 5 eq.) of EDC were dissolved in fresh and amine free DMF and subsequently added to (1). 246 mg (1.268 mmol, 4.6 eq.) of sulpho-NHS were dissolved in 10 mL of fresh and amine free DMF and added to the reaction mix. The reaction flask was covered with aluminium foil to protect the fluoresceine from light and the reaction mix was stirred over night (o.n.) at room temperature (RT) under argon atmosphere to avoid hydrolysis of the formed ester (2). The reaction progress was analyzed by TLC: THF:acetonitrile 3:1, R_{f} (2)=0.41. The solvent was evaporated and the product was washed 3 times with 10 mL acetonitrile and dissolved in 5 mL THF:acetonitrile (3:1), whereas brown precipitate was discarded. The ester (2) was purified employing silica-gel chromatography with gravity flow. The column (500 mm length, 20 mm diameter) was packed with 90 mL silica-gel (MP Silica 32-63, 60 Å, EcoChrom®) and equilibrated with THF:acetonitrile (3:1). The 5 mL reaction mix (2) was loaded onto the column and 20 fractions, 10 mL each, were collected and further analyzed by TLC (THF:acetonitrile, 3:1). Elution fractions 5-9 showed the majority of product (2) and were pooled. The solvent was evaporated and the product (2) (P2) dried under vacuum (<10⁻³ mbar). 225.6 mg=82% yield. P2 was analyzed and confirmed by mass spectrometry:
ESI-MS negative mode, m/z: 902 [M]⁻ (anion)

Formation of DEG(Fmoc)-SLIGRL P5

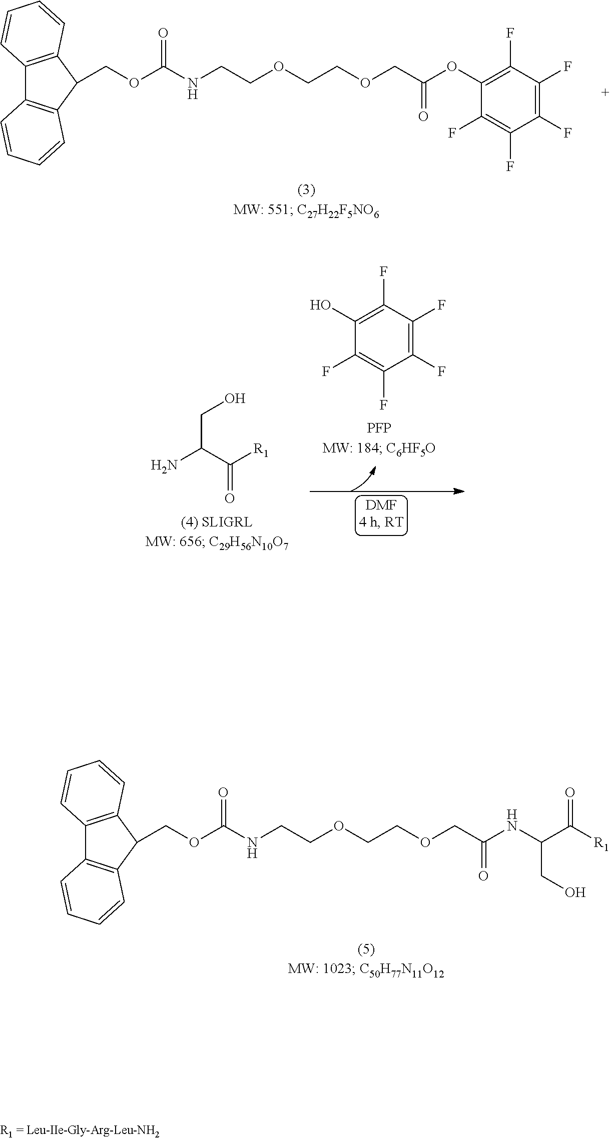

100 mg (152.4 μmol, 1 eq.) SLIGRL-NH2 (4) were dissolved in 3 mL of fresh and amine free DMF and transferred into a 50 mL reaction flask. 75.57 mg (137.16 μmol, 0.9 eq.) of Fmoc protected diethylenglycol-pentafluorophenol (3) were dissolved in 10 mL of fresh and amine free DMF and added to (4). The reaction mixture was filled up to 20 mL final reaction volume, with fresh and amine free DMF and stirred for 4 h at RT under argon atmosphere. The reaction progress was analysed by TLC: THF:acetonitrile 3:1, R_{f} (5)=0.63. The solvent was evaporated and the reaction mix concentrated to 5-10 mL. Product (5) was purified employing silica-gel chromatography with gravity flow. The column (500 mm length, 20 mm diameter) was packed with 60 mL silica-gel (MP Silica 32-63, 60 Å, EcoChrom®) and equilibrated with THF:acetonitrile (3:1). The 5-10 mL reaction mix (5) was loaded onto the column and washed with 100-140 mL THF:acetonitrile (3:1). Product (5) was then eluted with THF/MeOH (1:1). 10 fractions, 10 mL each, were collected and further analyzed by TLC (THF:acetonitrile, 3:1). Elution fractions 6-9 showed the majority of product (5) and were pooled. The solvent was evaporated and the product (5) (P5) was concentrated to 5 mL. P5 was analyzed and confirmed by mass spectrometry:
ESI-MS negative mode, m/z: 1022 [M−H]⁻
ESI-MS positive mode, m/z: 1024 [M+H]⁺
Tandem MS/MS of 1024, positive mode, m/z: 1024 [M+H]⁺, 894 [M+H, —H₂O,-Leu]⁺, 802 [M+H,-Fmoc]⁺, 738 [M+H, —H₂O,-Leu,-Arg]⁺, 681 [M+H, —H₂O,-Leu,-Arg,-Gly]⁺, 568 [M+H, —H₂O,-Leu,-Arg,-Gly,-Ile]⁺, 455 [M+H, —H₂O,-Leu,-Arg,-Gly,-Ile,-Leu]⁺

Formation of NH₂-DEG-SLIGRL P6

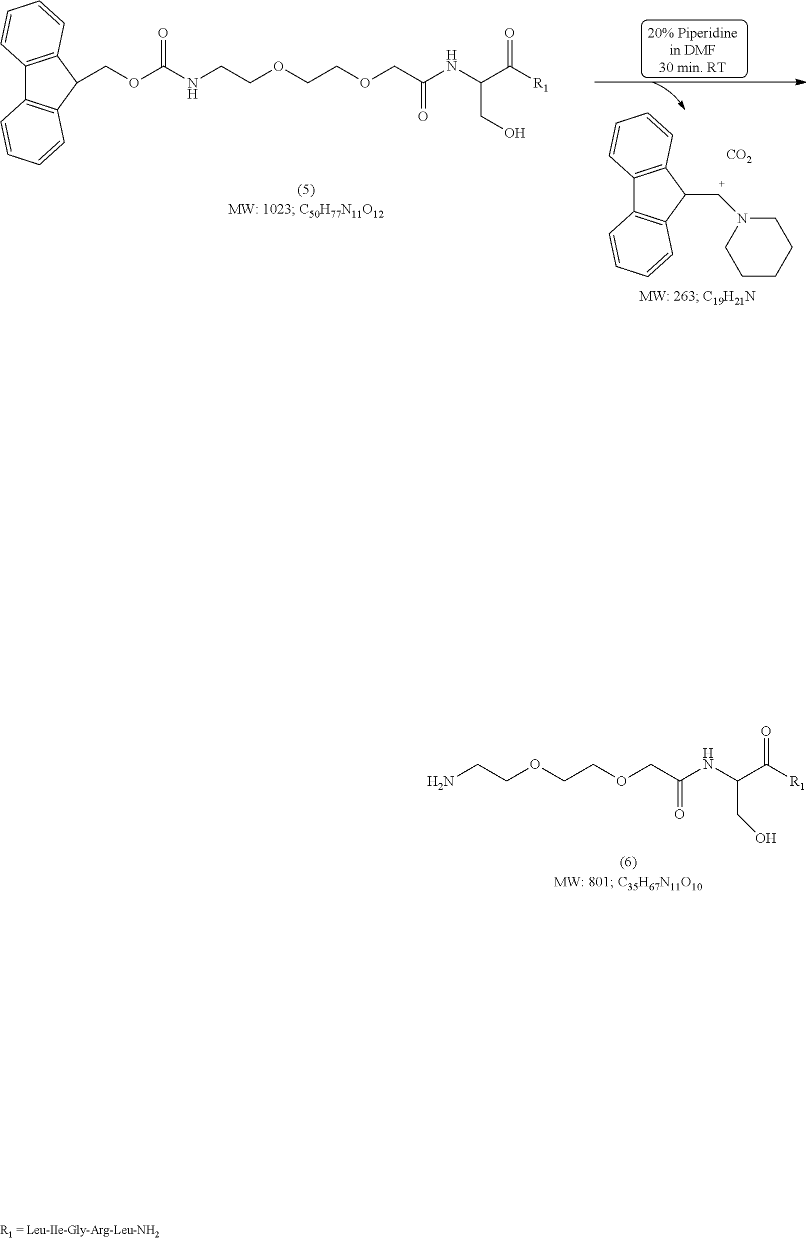

5 mL of fresh and amine free DMF were added to the 5 mL of product (5) and transferred into a 50 mL reaction flask. 5 mL of amine free piperidine and 10 mL of DMF were mixed and added to (5) to reach a final reaction volume of 25 mL containing 20% (v/v) piperidine. The reaction was stirred for 30 min at RT. The reaction progress was analyzed by TLC: THF:acetonitrile 3:1, R_{f} (5)=0.61, Rf (6)=0.81. The solvent was evaporated and reaction products were separated by organic phase extraction. The reaction products were washed 3 times with 20 mL acetonitrile and finally dissolved in 10 mL acetonitrile. The acetonitrile phase was washed 4 times with 10-20 mL n-heptane. n-heptane phases were pooled and washed 4 times with 20 mL acetonitrile. All n-heptane as well as acetonitrile phases were pooled and solvents were evaporated, respectively. TLC analysis confirmed the majority of product (6) to be present in the acetonitrile phase and impurities and possible side products to be mainly present in the n-heptane phase. Product (6) (P6) was dried under vacuum (<10⁻³ mbar). 60 μmol=39% yield. P6 was analyzed and confirmed by mass spectrometry:

ESI-MS negative mode, m/z: 800 [M−H]⁻
ESI-MS positive mode, m/z: 802 [M+H]⁺
Tandem MS/MS of 802, positive mode, m/z: 802 [M+H]⁺, 672 [M+H, −H₂O,-Leu]⁺, 516 [M+H, —H₂O,-Leu,-Arg,]⁺, 459 [M+H, 13 H₂O,-Leu,-Arg,-Gly]⁺

Formation of Fam5/6-Lysine(Fmoc)-DEG-SLIGRL P7

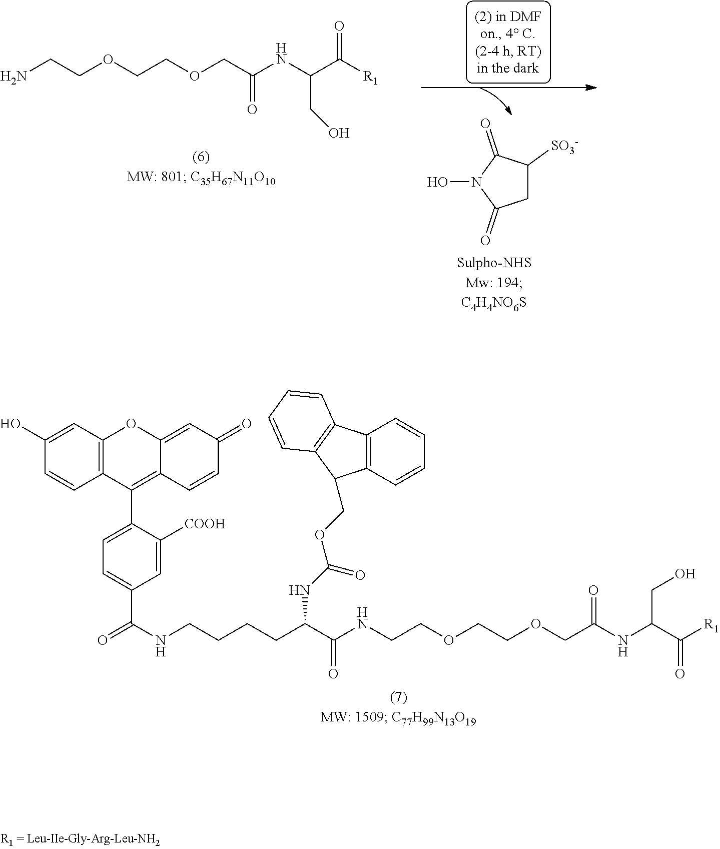

60 μmol (6) and 120 μmol (2) were dissolved in 10 mL fresh and amine free DMF (P6: 1.2 mM, P2: 2.4 mM final conc.) and transferred into a 50 mL reaction flask. The reaction flask was covered with aluminium foil to protect the fluoresceine from light and the reaction mix was stirred over night at room temperature under argon atmosphere to avoid hydrolysis of the ester (2).

The reaction progress was analyzed by TLC: THF:acetonitrile 3:1, R_{f} (7)=0.72, R_{f} (6)=0.85, R_{f} (2)=0.41. The solvent was evaporated and the reaction mix washed 3 times with 20 mL fresh and amine free DMF and dried under vacuum (<10⁻³ mbar). P7 was identified by mass spectrometry:

ESI-MS negative mode, m/z: 1508 [M−H]⁻
ESI-MS positive mode, m/z: 1510 [M+H]⁺
Tandem MS/MS of 1510, positive mode, m/z: 1510 [M+H]⁺, 1380 [M+H, —H₂O,-Leu]⁺, 1288 [M+H,-Fmoc]⁺, 1224 [M+H, 13 H₂O,-Leu,-Arg]⁺, 1167 [M+H, —H₂O ,-Leu,-Arg,-Gly]⁺, 1036 [M+H,-2H₂O,-Leu,-Arg,-Gly,-Ile]⁺, 923 [M+H,-2H₂O,-Leu,-Arg,-Gly,-Ile,-Leu]⁺

Formation of Fam5/6-Lysine-DEG-SLIGRL P8

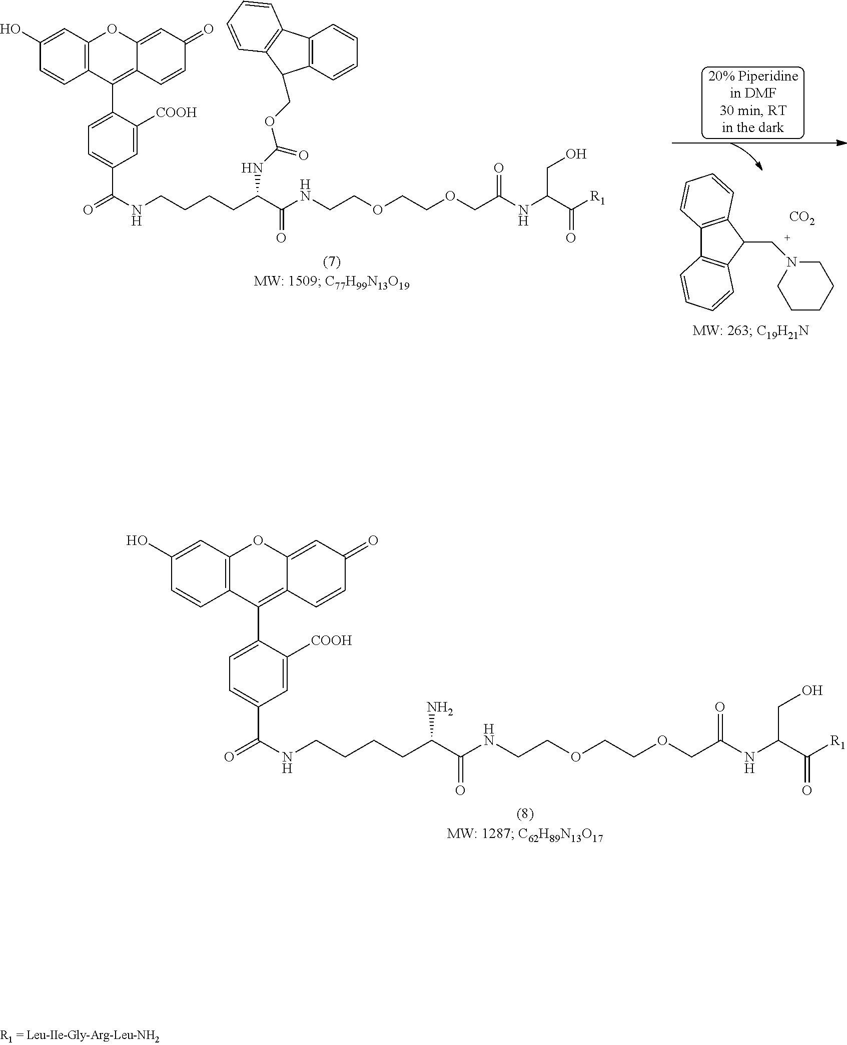

The reaction mix (7) was dissolved in 10 mL fresh and amine free DMF and transferred into a 50 mL reaction flask. 4 mL of amine free piperidine and 6 mL of DMF were mixed and added to the reaction mix (7) to reach a final reaction volume of 20 mL containing 20% (v/v) piperidine. The reaction flask was covered with aluminium foil to protect the fluoresceine from light and the reaction mix was stirred for 30 min at room temperature.

The reaction progress was analyzed by TLC: THF:acetonitrile 3:1, R_{f} (8)=0.05, R_{f} (6)=0.66. The solvent was evaporated and the reaction mix washed 3 times with 20 mL DMF and finally concentrated to 1 mL. 2 mL methanol was added and (8) was purified by preparative HPLC employing a Sphinx RP (C18/Phenol, 250/8, 5 μm, Macherey-Nagel) column with UV detection at 490 nm. The reaction products were separated on the column applying an elution gradient of acetonitrile in 0.1% acetic acid containing ddH₂O increasing from 20% acetonitrile to 100% within 30 min at a flow rate of 4 mL/min. The product (8) P8 was eluted at Rₜ=8.5 min (42.7% ACN). Elution fractions containing (8) were collected, pooled and the solvent was evaporated. Remaining acetic acid was removed by gel-filtration chromatography using a Sephadex G25 column (GE HealthCare) and ddH₂O as eluent. Aqueous (8) fractions were pooled and freeze-dried. 40 μmol=26.2% yield. P8 was analyzed and confirmed by mass spectrometry:

ESI-MS negative mode, m/z: 1286 [M−H]⁻
ESI-MS positive mode, m/z: 1288 [M+H]⁺, 1310 [M+Na]⁺
Tandem MS/MS of 1288, positive mode, m/z: 1288 [M+H]⁺, 1158 [M+H, —H₂O,-Leu]⁺, 1002 [M+H, —H₂O,-Leu,-Arg]⁺, 945 [M+H,—H₂O,-Leu,-Arg,-Gly]⁺, 814 [M+H,-2H₂O,-Leu,-Arg,-Gly,-Ile]⁺, 701 [M+H,-2H₂O,-Leu,-Arg,-Gly,-Ile,-Leu]⁺

In the synthesis method preferably employed in accordance with the present invention the critical step in the synthesis represents the conjugation of DEG, or another suitable linker, to the free N-terminus of the ligand, i.e. preferably amine, hydroxyl or thiol of a peptide, preferably amine of a serine such as in in SLIGRL-NH₂ (reaction (3)+(4) to (5), FIG. 2). Previously, it has been reported that exchanges of amino acids or other chemical modifications in the tethered ligand (TL) sequence lead to a significant reduction in PAR activation potential and subsequent signal transduction or even completely abolished the receptor activation potential [2,3]. For example, Maryanoff et al. observed that the mutation of leucine to alanine in position 2 of either the PAR2 agonist peptides SL₍₂₎IGRL-NH₂ (derived from rodent PAR2 TL) and SL₍₂₎IGKV-NH₂ (derived from human PAR2 TL) abolished the PAR2 activation potential of the ligand, whereas activation of PAR2 for the other five possible alanine mutations was still detected [3]. Moreover, acetylation of the serine in position 1 in both, S₍₁₎LIGRL-NH₂ and S₍₁₎LIGKV-NH₂ abolished the PAR2 activation potential, strongly indicating the importance of the free amine at the N-terminus of the PAR2 agonist peptide. However, the mutation of the serine to threonine retained ligand activity, whereas the exchange to phenylalanine lead to an inactive compound [3], suggesting that the correct positioning of a polar group, such as sulphydryl or hydroxyl group of the amino acid side chain, as in serine or threonine is important for ligand binding.

### Example 3: Design of Novel PAR Ligands by Simulating Molecular Receptor-Ligand Interactions (Ligand Docking Simulations)

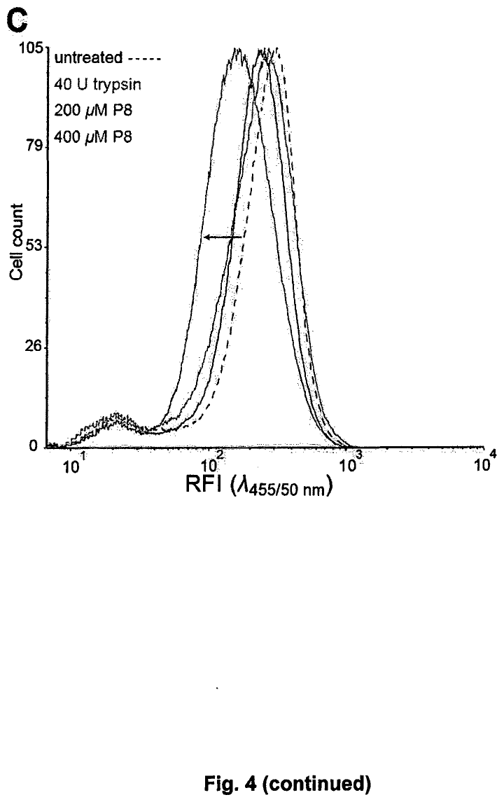

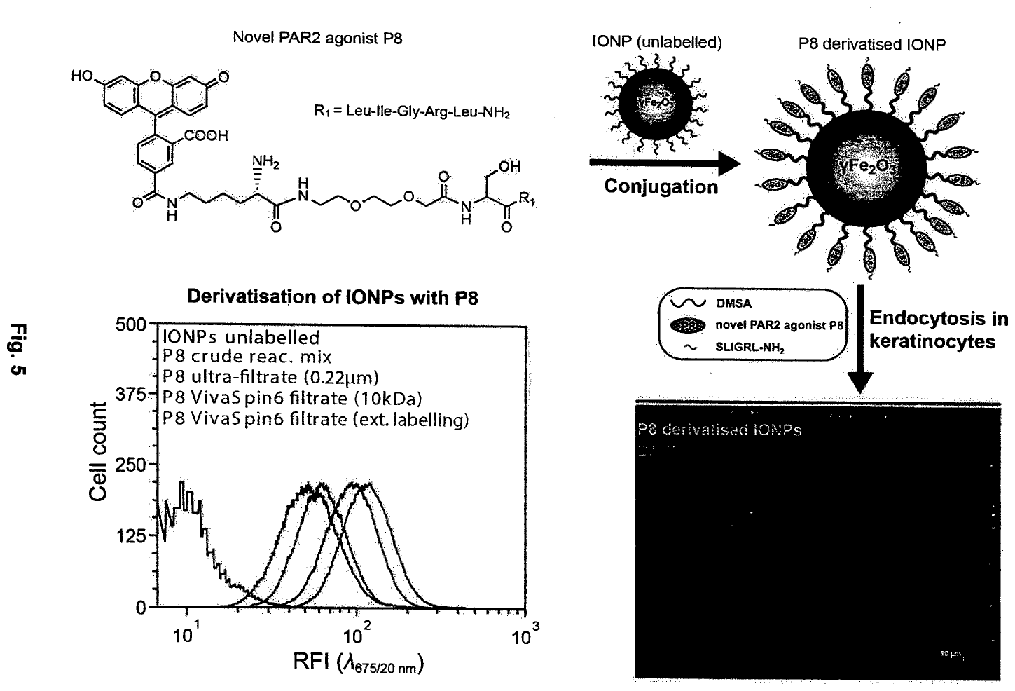

To investigate the receptor binding ability of PAR ligands, ligand-docking simulations were performed using the homology model of human PAR2 together with the intermediate compound P6 as well as the novel PAR2 agonist P8 (FIG. 3 A and B). The homology model of human PAR2 was generated using the crystal structure of human PAR1 (PDB 3VW7) as template structure. In both cases, PAR2-ligand complexes were obtained, in which the synthetic compounds P6 and P8 were localized at the proposed binding site, similar to that described by others [4,5]. Interestingly, it can be seen from FIG. 3 B that the novel PAR2 agonist P8 is oriented in a way that the sterically demanding fluorophore 5-FAM is located outside of the proposed binding pocket, allowing the biological active SLIGRL-NH₂ portion of the ligand to be located deep inside the receptor binding cleft. However, both, the well-defined orientation of P8 in the binding pocket and its sterically complex 5-FAM part might reduce the affinity to the receptor and thus also be the reason for the decreased PAR2-activation potential observed in the calcium mobilization assay (FIG. 4C).

This in silico molecular docking simulation represent a powerful tool to investigate potential intermolecular receptor-ligand interactions. In consequence, the model for P8 interactions with PAR2 described here provides the basis for the design of chemical modifications of PAR ligands, which then can be screened and tested for enhanced or modulated biological activities as described below, which covers selectivity and cell activation potential of such PAR agonists. In addition, selected biophysical methods, such as saturation transfer difference-nuclear magnetic resonance spectroscopy (STD-NMR), can be employed to empirically determine thermodynamic receptor-ligand interaction parameters, including association and dissociation constants as well as binding affinities [6].

### Example 4: Modified PAR Ligands Trigger Intracellular Calcium Mobilization

To test whether the modified PAR ligands such as the synthesis intermediates as well as the final compound P8 are capable of binding and activating PAR in vitro a calcium-based cell activation assay was established on the basis of the assay for measurement of intracellular Ca²⁺ mobilization described in Kelm et al., J. Exp. Med. 195 (2002), 1207-1213, which can be used directly on PAR, i.e. PAR2 expressing cells.

SOP: Measurement of Intracellular Ca²⁺ Mobilization
Thaw 1 aliquot of frozen (from liquid N₂) HEK293 cells (careful at RT)
Provide 10 mL of DMEM (Dulbecco's Modified Eagle Medium, phenol red free!) containing 10% FCS, antibiotics (50 μg/mL gentamicin final conc.) on a 10 cm cell culture plate
Add cells to the plate and grow them at 37° C., 8% CO₂ until 60-70% confluency (one cell layer on the plate)
Wash the cells 3× with 10 mM PBS (phosphate buffer saline), pH 7.4
Add 10 mL of fresh DMEM (phenol red free!) and carefully resuspend the cells
Count the cells employing a flow cytometer and dilute to 3×10⁵ cells/mL in DMEM (phenol red free!)
Pipette 1 ml of the diluted cell suspension in each well of a 24-well cell culture plate
Add 30 μL of Pluronic® F-127 (0.01% (v/v) final conc., P2443 Sigma-Aldrich) and 15 μL of Indo 1-AM ester in DMSO (15 μM final conc., I3261 Sigma-Aldrich) to the cell suspension
Incubate for 45 min at 37° C., 8% CO₂, in the dark
Henceforward keep the cells in the dark
Remove cells from the culture plate and subsequently wash 1× with 15 mL of HEPES buffer (100 mM HEPES, pH 7.4) to remove excess Indo 1-AM ester and resuspend cells in 1 mL HEPES buffer (100 mM HEPES, pH 7.4)
Add 10 μL propidium iodide (PI: final conc. 100 ng/mL, P4864 Sigma-Aldrich) to the cell suspension and immediately perform flow cytometry analysis
Perform flow cytometry analysis using the appropriate laser/filter sets (CyFlow® Cube 8 Sysmex):

Indo 1-AM: λ_{excitation,max} 345 nm, λ_{emission,max} 400 nm (Ca²⁺ bound), λ_{emission,max} 475 nm (Ca²⁺ free), (excitation: UV-LED, emission: FL4 channel, 455/50 nm)

PI: λ_{excitation,max} 535 nm (DNA-bound), λ_{emission,max} 617 nm (DNA-bound), (excitation: 488 nm laser, emission: FL2 channel, 590/50 nm)
For PAR-2 activation, add the appropriate amount of activator (SLIGRL: 50 μM final conc., trypsin: 40 Units (TRYPSEQM-RO Roche), P6 or P8: 100-200 μM or 200-400 μM respectively) to the cell suspension (containing 15 nmol Indo 1-AM ester) and immediately perform flow cytometry analysis
Determine the decrease in relative fluorescence intensity (due to the shift from unbound towards calcium bound Indo 1-AM complexes) after PAR mediated activation of HEK293 cells

As described above, activation of PARs leads to the initiation of a variety of signal transduction pathways and downstream signaling. One consequence of PAR activation presents the intracellular calcium mobilization from storage reservoirs of the endoplasmic reticulum (ER) and mitochondria into the cytosol. These changes in intracellular free calcium concentrations can be detected using a calcium sensitive fluorescence probe, which exhibits a concentration dependent change in spectral response upon calcium binding [7]. In the present assay the Indo-1 AM fluorophore was used, which is an esterified derivative of Indo-1 and thus able to penetrate the cell membrane. Upon uptake into the cytoplasm, present esterases will transfer the ester into the carboxylate, which is then trapped in the cell due to its obtained negative charges. A shift in Indo-1 AM emission from 480 nm (calcium free Indo-1 AM) to 400 nm (calcium bound Indo-1 AM) can be detected employing a flow cytometer, when bound to free calcium.

In brief, we preincubated PAR2-expressing HEK293 (human embryonic kidney) cells with Indo-1 AM and subsequently removed excess dye by washing the cells thoroughly. PAR2 activation was initiated either with trypsin, free PAR2 agonist peptide SLIGRL-NH2 or with our intermediate compound P6 as well as the synthetic agonist P8 right before flow cytometry analysis (FIG. 4). Both, P6 and P8 induced a shift in relative fluorescence intensity (RFI) of the cells relative to the untreated sample (FIG. 4 A-C), demonstrating their capability to activate PAR2 triggering subsequent intracellular calcium mobilization. However, the relative potential of PAR2 activation decreased from SLIGRL-NH₂, to P6 and further to P8 (FIG. 4 A-C), which might represent a direct consequence due to the N-terminal derivatization of SLIGRL-NH₂ as discussed above.

### Example 5: PAR Ligand Mediates Internalization of Conjugated Biological Entity of Interest into a Target Cell

By way of example with the novel PAR2 agonist P8, the potential of PAR ligand-conjugates of the present invention to mediate the internalization of an entity of interest, here a nanoparticle when immobilized onto its surface was tested. Therefore, as proof-of concept iron oxide nanoparticles (IONP) [8,9] were used as model particles for the derivatization with the PAR ligand P8 and subsequent endocytosis.

Formation of P8-Labelled IONPs

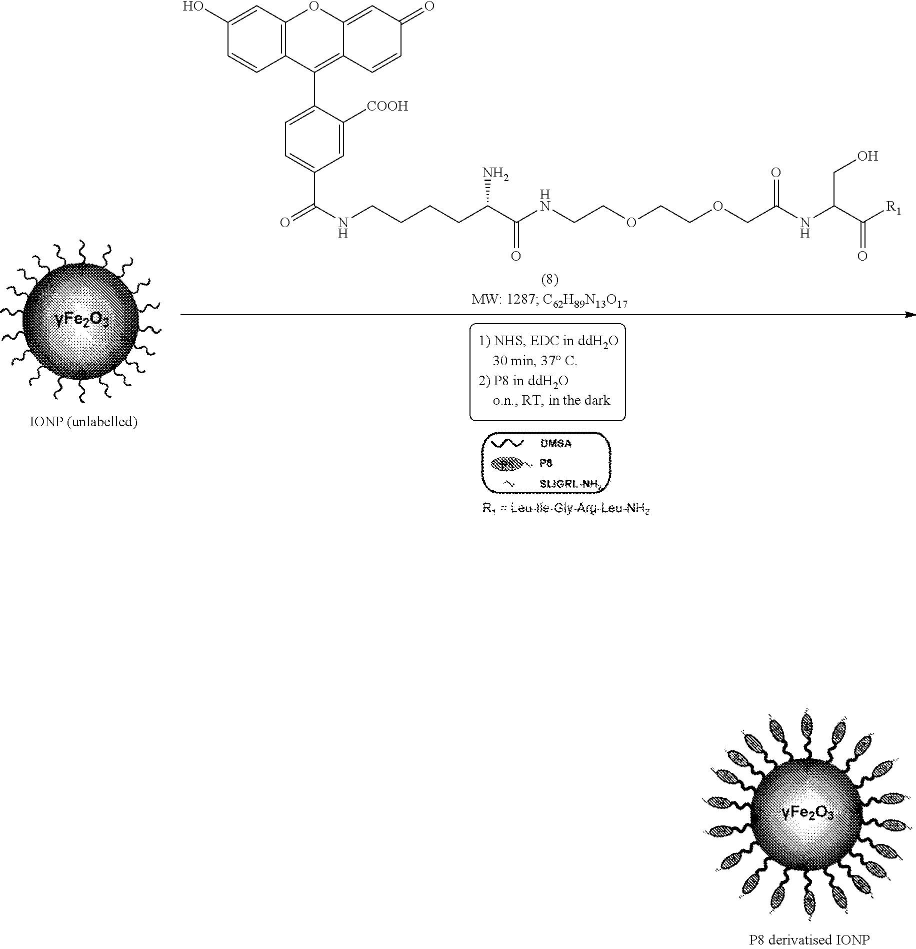

561.8 μL of DMSA derivatized IONP suspension (20 μmol iron content, IONPs were pipetted into a 1.5 mL Eppendorf reaction tube and mixed with 413.75 μL of ddH₂O. 20 μL of a 10 mM EDC (200 mmol) stock solution and 1 μL of a 500 mM NHS (500 nmol) stock solution were added to the IONP suspension and gently mixed. The reaction mix was incubated on a thermoblock for 30 min at 37° C. After the incubation 3.45 μL of a 29 mM P8 (100 nmol) stock solution were added to the suspension. The reaction tube was covered with aluminium foil and the reaction mix was further incubated on a rotation wheel (12 rpm) over night at RT. The reaction mix was filtered through a 22 μm filter (3000×g, 5 min, 22° C.). The flow-through containing the P8 derivatized IONPs was collected and 1 mL ddH₂O was added on top of the filter and centrifuged again (3000×g, 5 min, 22° C.). Both flow-through were pooled and transferred into a VivaSpin6® filtration unit (10 kDa cut off, Sartorius) and centrifuged for 5 min at 1000×g, 22° C. The flow-through was discarded and 6 mL ddH₂O was added on top of the Vivaspin6 unit and centrifuged again (1000×g, 15 min, 22° C.). This washing step was repeated three times. The washed P8 derivatized IONP suspension was concentrated to 1 mL and characterized by flow cytometry.

SOP: PAR2 Mediated Endocytosis Assay
Culture HaCaT (cultured human keratinocytes) cells in DMEM (Dulbecco's Modified Eagle Medium, phenol red free!) supplemented with 10% (w/v) FCS and antibiotics (gentamicin: 50 μg/mL final conc.) in a 10 cm cell culture plate at a confluency (one cell layer on the plate) of 60-70%
Count the cells and dilute to 1-2×10⁴ cells/mL in DMEM (phenol red free!)
Incubate 1 mL of the diluted cell suspension in each well of a 24-well cell culture plate supplemented with 10% (w/v) FCS (containing gentamicin at a final conc. of 50 μg/mL) on a glass coverslip for 24 h at 37° C., 5% CO₂
Discard the supernatant and carefully wash the cells 3 times with 500 μL DMEM (phenol red free, without FCS!)
Incubate the cells for 24 h at 37° C., 5% CO₂ (DMEM without serum addition, phenol red free only!)
Add CaCl₂ solution to reach a final concentration of 1.5 mM in the medium when using Ca²⁺-free medium (induction of cell differentiation)
For PAR2 activation

with Trypsin:
Add 500 μL trypsin (T4549 Sigma-Aldrich) in PBS (0.05% (w/v) final conc.) to the cells and incubate for 45 sec at 37° C., 5% CO₂
Stop the treatment by washing the cells 3× with DMEM containing 10% (w/v) FCS, to inhibit trypsin activity and prevent subsequent cell damage, and 1× with FCS-free DMEM
Add particles, for example OregonGreen® labelled IONPs (iron oxide nano particles [1,2], 100-150 nmol final iron content in solution) as model particles to monitor endocytosis, in DMEM to the cells and incubate for 2 h at 37° C., 5% CO₂

with SLIGRL:
Add 500 μL of a solution containing SLIGRL (500 nM final conc.) and particles, for example OregonGreen® labelled IONPs (100-150 nmol final iron content in solution), as model particles to monitor endocytosis, in DMEM to the cells and incubate for 2 h at 37° C., 5% CO₂

with P8-Labelled IONPs:
Add P8-labelled IONPs in DMEM to the cells (100-150 nmol final iron content in solution) and incubate for 2 h at 37° C., 5% CO₂
Wash the cells 3× with 1 mL 10 mM PBS (phosphate buffer saline), pH 7.4
Determine phagocytosis efficiency employing fluorescence microscopy (ApoTome.2, Zeiss):
   DAPI staining: λ_{excitation,max} 358 nm (DNA-bound), λ_{emission,max} 461 nm (DNA-bound)
   OregonGreen® labeled IONPs: λ_{excitation,max} 501 nm, λ_{emission,max} 526 nm
   P8-labelled IONPs: λ_{excitation,max} 495 nm, λ_{emission,max} 520 nm

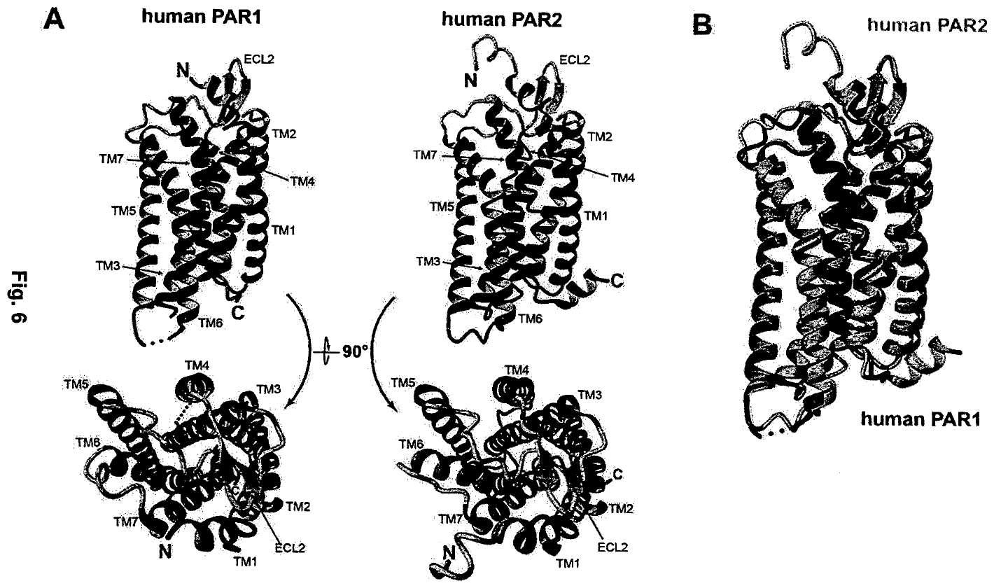

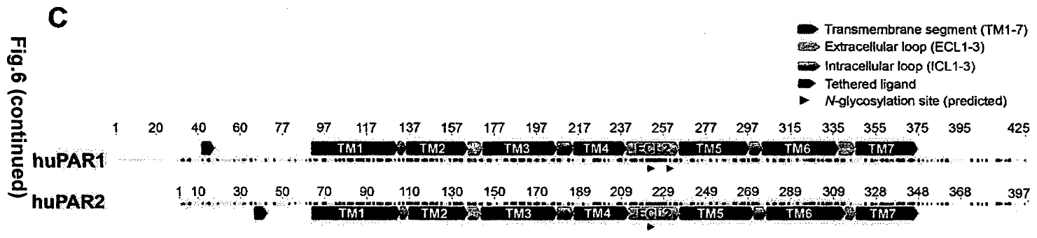

An endocytosis assay was used to test whether the PAR ligand-conjugate, i.e. PAR2 agonist P8 enhances the uptake of IONPs into PAR, here PAR2 expressing keratinocytes, when immobilized to the surface of the IONP. In brief, cultured keratinocytes were serum-starved for 24 hours to trigger translocation of PAR receptors to the cell surface [10]. P8-conjugated IONPs were incubated for 2 hours together with the keratinocytes. After the incubation, cells were washed thoroughly for several times and internalization of the modified IONPs was analyzed employing fluorescent microscopy (FIG. 5, lower right corner). FIG. 5 illustrates the successful uptake of our P8 modified IONPs (green dots) into cultured keratinocytes (cell nuclei are stained with DAPI: 4′,6-diamidine-2′-phenylindole). It should be noted that the green dots do not represent single IONPs, since the signal of one fluorescently labelled IONP is far too weak to be detected with this method. In fact, only accumulated fluorescently labelled IONPs produce such relatively strong signals, also depending on the nature of the conjugated fluorophore. Thus, the internalized PAR ligand modified IONPs are located in microvesicles, such as endosomes or lysosomes [11].

### REFERENCES

1. McGuire J J, Saifeddine M, Triggle C R, Sun K, Hollenberg M D. 2-furoyl-LIGRLO-amide: a potent and selective proteinase-activated receptor 2 agonist. J. Pharmacol. Exp. Ther. American Society for Pharmacology and Experimental Therapeutics; 2004; 309:1124-31.

2. Jiang Y, Yau M-K, Kok W M, Lim J, Wu K-C, Liu L, et al. Biased signaling by agonists of protease activated receptor 2. ACS Chem. Biol. 2017; acschembio.6b01088-10.

3. Maryanoff B E, Santulli R J, McComsey D F, Hoekstra W J, Hoey K, Smith C E, et al. Protease-Activated Receptor-2 (PAR-2): Structure-function study of receptor activation by diverse peptides related to tethered-ligand epitopes. Arch. Biochem. Biophys. 2001; 386:195-204.

4. Kakarala K K, Jamil K, Devaraji V. Structure and putative signaling mechanism of Protease Activated Receptor 2 (PAR2)—A promising target for breast cancer. Journal of Molecular Graphics and Modelling. Elsevier Inc; 2014; 53:179-99.

5. Perry S R, Xu W, Wirija A, Lim J, Yau M-K, Stoermer M J, et al. Three homology models of PAR2 derived from different templates: Application to antagonist discovery. J. Chem. Inf. Model. 2015; 55:1181-91.

6. Meyer B, Peters T. NMR spectroscopy techniques for screening and identifying ligand binding to protein receptors. Angew. Chem. Int. Ed. Engl. 2003; 42:864-90.

7. Valet G, Raffael A, Rüssmann L. Determination of intracellular calcium in vital cells by flow-cytometry. Naturwissenschaften. 1985; 72:600-2.

8. Massart R. Preparation of aqueous magnetic liquids in alkaline and acidic media. IEEE T MAGN. 1981; 17:1247-8.

9. Ruiz A, Morais P C, Bentes de Azevedo R, Lacava ZGM, Villanueva A, del Puerto Morales M. Magnetic nanoparticles coated with dimercaptosuccinic acid: development, characterization, and application in biomedicine. J Nanopart Res. 2014; 16:279-21.

10. Déry O, Thoma M S, Wong H, Grady E F, Bunnett N W. Trafficking of proteinase-activated receptor-2 and beta-arrestin-1 tagged with green fluorescent protein. beta-Arrestin-dependent endocytosis of a proteinase receptor. J. Biol. Chem. 1999; 274:18524-35.

11. Belleudi F, Purpura V, Scrofani C, Persechino F, Leone L, Torrisi M R. Expression and signaling of the tyrosine kinase FGFR2b/KGFR regulates phagocytosis and melanosome uptake in human keratinocytes. FASEB J. 2011; 25:170-81.

12. Alexander S P H, Benson H E, Faccenda E, Pawson A J, Sharman J L, Spedding M, et al. The Concise Guide to PHARMACOLOGY 2013/14: G protein-coupled receptors. Br. J. Pharmacol. 2013; 170:1459-581.

13. Rasmussen U B, Vouret-Craviari V, Jallat S, Schlesinger Y, Pagès G, Pavirani A, et al. cDNA cloning and expression of a hamster alpha-thrombin receptor coupled to Ca2+ mobilization. FEBS Lett. 1991; 288:123-8.

14. Vu T K, Hung D T, Wheaton V I, Coughlin S R. Molecular cloning of a functional thrombin receptor reveals a novel proteolytic mechanism of receptor activation. Cell. 1991; 64:1057-68.

15. Okada T, Sugihara M, Bondar A-N, Elstner M, Entel P, Buss V. The retinal conformation and its environment in rhodopsin in light of a new 2.2 A crystal structure. Journal of Molecular Biology. 2004; 342:571-83.

16. Thompson A A, Liu W, Chun E, Katritch V, Wu H, Vardy E, et al. Structure of the nociceptin/orphanin FQ receptor in complex with a peptide mimetic. Nature. 2012; 485:395-9.

17. Vu T K, Wheaton V I, Hung D T, Charo I, Coughlin S R. Domains specifying thrombin-receptor interaction. Nature. 1991; 353:674-7.

18. Nystedt S, Emilsson K, Wahlestedt C, Sundelin J. Molecular cloning of a potential proteinase activated receptor. Proc. Natl. Acad. Sci. U.S.A. National Academy of Sciences; 1994; 91:9208-12.

19. Nystedt S, Emilsson K, Larsson A K, Strömbeck B, Sundelin J. Molecular cloning and functional expression of the gene encoding the human proteinase-activated receptor 2. Eur. J. Biochem. 1995; 232:84-9.

20. Nystedt S, Ramakrishnan V, Sundelin J. The proteinase-activated receptor 2 is induced by inflammatory mediators in human endothelial cells. Comparison with the thrombin receptor. J. Biol. Chem. 1996; 271:14910-5.

21. Macfarlane S R, Seatter M J, Kanke T, Hunter G D, Plevin R. Proteinase-activated receptors. Pharmacol. Rev. 2001; 53:245-82.

22. Adams M N, Ramachandran R, Yau M-K, Suen J Y, Fairlie D P, Hollenberg M D, et al. Structure, function and pathophysiology of protease activated receptors. Pharmacol. Ther. 2011; 130:248-82.

23. Molino M, Barnathan E S, Numerof R, Clark J, Dreyer M, Cumashi A, et al. Interactions of mast cell tryptase with thrombin receptors and PAR-2. J. Biol. Chem. 1997; 272:4043-9.

24. Camerer E, Huang W, Coughlin S R. Tissue factor- and factor X-dependent activation of protease-activated receptor 2 by factor VIIa. Proc. Natl. Acad. Sci. U.S.A. 2000; 97:5255-60.

25. Riewald M, Kravchenko V V, Petrovan R J, O'Brien P J, Brass L F, Ulevitch R J, et al. Gene induction by coagulation factor Xa is mediated by activation of protease-activated receptor 1. Blood. 2001; 97:3109-16.

26. Suidan H S, Bouvier J, Schaerer E, Stone S R, Monard D, Tschopp J. Granzyme A released upon stimulation of cytotoxic T lymphocytes activates the thrombin receptor on neuronal cells and astrocytes. Proc. Natl. Acad. Sci. U.S.A. National Academy of Sciences; 1994; 91:8112-6.

27. Takeuchi T, Harris J L, Huang W, Yan K W, Coughlin S R, Craik C S. Cellular localization of membrane-type serine protease 1 and identification of protease-activated receptor-2 and single-chain urokinase-type plasminogen activator as substrates. J. Biol. Chem. American Society for Biochemistry and Molecular Biology; 2000; 275:26333-42.

28. Mize G J, Wang W, Takayama T K. Prostate-specific kallikreins-2 and -4 enhance the proliferation of DU-145 prostate cancer cells through protease-activated receptors-1 and -2. Mol. Cancer Res. American Association for Cancer Research; 2008; 6:1043-51.

29. Ramsay A J, Reid J C, Adams M N, Samaratunga H, Dong Y, Clements J A, et al. Prostatic trypsin-like kallikrein-related peptidases (KLKs) and other prostate-expressed tryptic proteinases as regulators of signalling via proteinase-activated receptors (PARs). Biol. Chem. 2008; 389:653-68.

30. Oikonomopoulou K, Hansen K K, Saifeddine M, Tea I, Blaber M, Blaber S I, et al. Proteinase-activated receptors, targets for kallikrein signaling. J. Biol. Chem. American Society for Biochemistry and Molecular Biology; 2006; 281:32095-112.

31. Boire A, Covic L, Agarwal A, Jacques S, Sherifi S, Kuliopulos A. PAR1 is a matrix metalloprotease-1 receptor that promotes invasion and tumorigenesis of breast cancer cells. Cell. 2005; 120:303-13.

32. Connolly A J, Ishihara H, Kahn M L, Farese R V, Coughlin S R. Role of the thrombin receptor in development and evidence for a second receptor. Nature. 1996; 381:516-9.

33. Xu W F, Andersen H, Whitmore T E, Presnell S R, Yee D P, Ching A, et al. Cloning and characterization of human protease-activated receptor 4. Proc. Natl. Acad. Sci. U.S.A. 1998; 95:6642-6.

34. Nieman M T, Schmaier A H. Interaction of thrombin with PAR1 and PAR4 at the thrombin cleavage site. Biochemistry. 2007; 46:8603-10.

35. Yau M-K, Liu L, Fairlie D P. Toward drugs for protease-activated receptor 2 (PAR2). J. Med. Chem. 2013; 56:7477-97.

36. Nystedt S, Larsson A K, Aberg H, Sundelin J. The mouse proteinase-activated receptor-2 cDNA and gene. Molecular cloning and functional expression. J. Biol. Chem. 1995; 270:5950-5.

37. Bohm S K, Khitin L M, Grady E F, Aponte G, Payan D G, Bunnett N W. Mechanisms of desensitization and resensitization of proteinase-activated receptor-2. J. Biol. Chem. 1996; 271:22003-16.

38. Böhm S K, Kong W, Bromme D, Smeekens S P, Anderson D C, Connolly A, et al. Molecular cloning, expression and potential functions of the human proteinase-activated receptor-2. Biochem. J. Portland Press Ltd; 1996; 314 (Pt 3):1009-16.

39. Vergnolle N. Protease-activated receptors as drug targets in inflammation and pain. Pharmacol. Ther. 2009; 123:292-309.

40. Déry O, Corvera C U, Steinhoff M, Bunnett N W. Proteinase-activated receptors: novel mechanisms of signaling by serine proteases. Am. J. Physiol. 1998; 274:C1429-52.

41. Zhao P, Lieu T, Barlow N, Sostegni S, Haerteis S, Korbmacher C, et al. Neutrophil elastase activates Protease-Activated Receptor-2 (PAR2) and transient receptor potential vanilloid 4 (TRPV4) to cause inflammation and pain. Journal of Biological Chemistry. American Society for Biochemistry and Molecular Biology; 2015; 290:13875-87.

42. Kimple A J, Bosch D E, Giguère P M, Siderovski D P. Regulators of G-protein signaling and their Ga substrates: promises and challenges in their use as drug discovery targets. Christopoulos A, editor. Pharmacol. Rev. American Society for Pharmacology and Experimental Therapeutics; 2011; 63:728-49.

43. Vouret-Craviari V, Van Obberghen-Schilling E, Rasmussen U B, Pavirani A, Lecocq J P, Pouysségur J. Synthetic alpha-thrombin receptor peptides activate G protein-coupled signaling pathways but are unable to induce mitogenesis. Mol. Biol. Cell. American Society for Cell Biology; 1992; 3:95-102.

44. Vouret-Craviari V, Van Obberghen-Schilling E, Scimeca J C, Van Obberghen E, Pouysségur J. Differential activation of p44mapk (ERK1) by alpha-thrombin and thrombin-receptor peptide agonist. Biochem. J. Portland Press Ltd; 1993; 289 (Pt 1):209-14.

45. Ghosh E, Kumari P, Jaiman D, Shukla A K. Methodological advances: the unsung heroes of the GPCR structural revolution. Nat. Rev. Mol. Cell Biol. 2015; 16:69-81.

46. Benovic J L, DeBlasi A, Stone W C, Caron M G, Lefkowitz R J. Beta-adrenergic receptor kinase: primary structure delineates a multigene family. Ullstein Mosby. 1989; 246:235-40.

47. Lohse M J, Benovic J L, Codina J, Caron M G, Lefkowitz R J. beta-Arrestin: a protein that regulates beta-adrenergic receptor function. Ullstein Mosby. 1990; 248:1547-50.

48. Suen J Y, Cotterell A, Lohman R J, Lim J, Han A, Yau M K, et al. Pathway-selective antagonism of proteinase activated receptor 2. Br. J. Pharmacol. 2014; 171:4112-24.

49. DeFea KA, Zalevsky J, Thoma M S, Déry O, Mullins R D, Bunnett N W. beta-arrestin-dependent endocytosis of proteinase-activated receptor 2 is required for intracellular targeting of activated ERK1/2. J. Cell Biol. 2000; 148:1267-81.

50. Ayoub M A, Pin J-P. Interaction of Protease-Activated Receptor 2 with G proteins and β-arrestin 1 studied by bioluminescence resonance energy transfer. Front Endocrinol (Lausanne). 2013; 4:196.

51. Noorbakhsh F, Vergnolle N, Hollenberg M D, Power C. Proteinase-activated receptors in the nervous system. Nat. Rev. Neurosci. 2003; 4:981-90.

52. Coughlin S R. Protease-activated receptors in hemostasis, thrombosis and vascular biology. J. Thromb. Haemost. Blackwell Science Inc; 2005; 3:1800-14.

53. Gao L, Chao L, Chao J. A novel signaling pathway of tissue kallikrein in promoting keratinocyte migration: activation of proteinase-activated receptor 1 and epidermal growth factor receptor. Exp. Cell Res. 2010; 316:376-89.

54. Vergnolle N. Clinical relevance of proteinase activated receptors (pars) in the gut. Gut. BMJ Publishing Group; 2005; 54:867-74.

55. Seiberg M. Keratinocyte-melanocyte interactions during melanosome transfer. Pigment Cell Res. 2001; 14:236-42.

56. Vergnolle N. Protease-activated receptors and inflammatory hyperalgesia. Mem Inst Oswaldo Cruz. 2005; 100 Suppl 1:173-6.

57. Ruf W, Mueller B M. Thrombin generation and the pathogenesis of cancer. Semin. Thromb. Hemost. Copyright © 2006 by Thieme Medical Publishers, Inc., 333 Seventh Avenue, New York, N.Y. 10001, USA; 2006; 32 Suppl 1:61-8.

58. Soreide K, Janssen E A, Körner H, Baak J P A. Trypsin in colorectal cancer: molecular biological mechanisms of proliferation, invasion, and metastasis. J. Pathol. John Wiley & Sons, Ltd; 2006; 209:147-56.

59. D'Andrea M R, Derian C K, Santulli R J, Andrade-Gordon P. Differential expression of protease-activated receptors-1 and -2 in stromal fibroblasts of normal, benign, and malignant human tissues. Am. J. Pathol. Elsevier; 2001; 158:2031-41.

60. Freund-Michel V, Frossard N. Inflammatory conditions increase expression of protease-activated receptor-2 by human airway smooth muscle cells in culture. Fundam Clin Pharmacol. Blackwell Publishing Ltd; 2006; 20:351-7.

61. Ossovskaya V S, Bunnett N W. Protease-activated receptors: contribution to physiology and disease. Physiol. Rev. American Physiological Society; 2004; 84:579-621.

62. Hansen K K, Oikonomopoulou K, Baruch A, Ramachandran R, Beck P, Diamandis E P, et al. Proteinases as hormones: targets and mechanisms for proteolytic signaling. Biol. Chem. 2008; 389:971-82.

63. Ramachandran R, Hollenberg M D. Proteinases and signalling: pathophysiological and therapeutic implications via PARs and more. Br. J. Pharmacol. Blackwell Publishing Ltd; 2008; 153 Suppl 1:S263-82.

64. Ohlsson K, Olsson I. The extracellular release of granulocyte collagenase and elastase during phagocytosis and inflammatory processes. Scand J Haematol. 1977; 19:145-52.

65. Molino M, Blanchard N, Belmonte E, Tarver A P, Abrams C, Hoxie J A, et al. Proteolysis of the human platelet and endothelial cell thrombin receptor by neutrophil-derived cathepsin G. J. Biol. Chem. 1995; 270:11168-75.

66. Nakanishi-Matsui M, Zheng Y W, Sulciner D J, Weiss E J, Ludeman M J, Coughlin S R. PAR3 is a cofactor for PAR4 activation by thrombin. Nature. 2000; 404:609-13.

67. Hansen K K, Saifeddine M, Hollenberg M D. Tethered ligand-derived peptides of proteinase-activated receptor 3 (PAR3) activate PAR1 and PAR2 in Jurkat T cells. Immunology. Blackwell Science Ltd; 2004; 112:183-90.

68. Ahn H S, Foster C, Boykow G, Arik L, Smith-Torhan A, Hesk D, et al. Binding of a thrombin receptor tethered ligand analogue to human platelet thrombin receptor. Mol. Pharmacol. 1997; 51:350-6.

69. Matsoukas J, Hollenberg M D, Mavromoustakos T, Panagiotopoulos D, Alexopoulos K, Yamdagni R, et al. Conformational analysis of the thrombin receptor agonist peptides SFLLR and SFLLR-NH2 by NMR: evidence fora cyclic bioactive conformation. J. Protein Chem. 1997; 16:113-31.

70. Nose T, Fujita T, Nakajima M, Inoue Y, Costa T, Shimohigashi Y. Interaction mode of the phe-phenyl group of thrombin receptor-tethered ligand SFLLRNP in receptor activation. J. Biochem. 1998; 124:354-8.

71. McComsey D F, Hecker L R, Andrade-Gordon P, Addo M F, Maryanoff B E. Macrocyclic hexapeptide analogues of the thrombin receptor (PAR-1) activation motif SFLLRN. Bioorg. Med. Chem. Lett. 1999; 9:255-60.

72. Scarborough R M, Naughton M A, Teng W, Hung D T, Rose J, Vu T K, et al. Tethered ligand agonist peptides. Structural requirements for thrombin receptor activation reveal mechanism of proteolytic unmasking of agonist function. J. Biol. Chem. 1992; 267:13146-9.

73. Yau M-K, Liu L, Suen J Y, Lim J, Lohman R-J, Jiang Y, et al. PAR2 modulators derived from GB88. ACS Med. Chem. Lett. 2016; 7:1179-84.

74. Yau M-K, Suen J Y, Xu W, Lim J, Liu L, Adams M N, et al. Potent small agonists of Protease Activated Receptor 2. ACS Med. Chem. Lett. 2016; 7:105-10.

75. Yau M-K, Lim J, Liu L, Fairlie D P. Protease Activated Receptor 2 (PAR2) modulators: a patent review (2010-2015). Expert Opinion on Therapeutic Patents. Taylor & Francis; 2016; 26:471-83.

76. Xu W, Lim J, Goh C-Y, Suen J Y, Jiang Y, Yau M-K, et al. Repurposing registered drugs as antagonists for Protease-Activated Receptor 2. J. Chem. Inf. Model. 2015; 55:2079-84.

What is claimed is:

1. A conjugate capable of triggering target specific cellular uptake of an entity of interest comprising:
(a) a protease activated receptor (PAR)-binding ligand; and covalently attached thereto
(b) a linker molecule which provides the ability to be chemically linked to the entity of interest; and optionally
(c) the entity of interest conjugated to the linker.

2. The conjugate of claim 1, wherein the linker is attached to the ligand by a spacer molecule, preferably diethylenglycol (DEG) or polyethylenglycol (PEG).

3. The conjugate of claim 1, wherein the PAR is PAR2, preferably human PAR and PAR2, respectively.

4. The conjugate of claim 1, wherein the ligand is a PAR activating peptide or derivative thereof, preferably wherein the peptide has a serine at the N-terminus and/or the carboxylate at the C-terminus modified to a primary amide.

5. The conjugate of claim 4, wherein the linker is directly or indirectly attached to the N-terminus of the peptide, preferably at the free amine of the serine.

6. The conjugate of claim 4, wherein the PAR activating peptide comprises the amino acid sequence SLIGRL (SEQ ID NO: 1), SLIGKV (SEQ ID NO: 3), or a derivative thereof, preferably SLIGRL-NH₂ or SLIGKV-NH₂.

7. The conjugate of claim 1, wherein the linker molecule is lysine.

8. The conjugate of claim 1, wherein the linker comprises a detectable label, preferably a fluorophore such as 6-carboxyfluorescein (6-FAM) and 5-carboxyfluorescein (5-FAM) or a mixture thereof.

9. The conjugate of claim 1, wherein the entity of interest is a diagnostic, cosmetic or therapeutic agent, a cell, a micro-vesicle or other nano- or micro-particle, preferably iron oxide nanoparticle (IONP).

10. A PAR ligand comprising or consisting of a PAR activating peptide or derivative thereof such as defined in claim 4, which is modified with a functional moiety at its N-terminus, preferably at a serine, preferably wherein the functional moiety is spacer or linker molecule.

11. A composition or kit comprising the conjugate of claim 1, preferably which is
(i) a pharmaceutical composition and comprises a pharmaceutically acceptable carrier; or
(ii) is a diagnostic composition and optionally comprises suitable means for detection of the ligand,
preferably wherein the conjugate comprises the entity of interest.

12. A conjugate of claim 1 for use in the treatment, diagnosis or monitoring of a disease or condition related to the over-expression of PAR in a cell, preferably wherein the disease is cancer or for the modulation of a cell expressing PAR in vitro.

13. Use of a PAR ligand, linker, spacer, detectable label, or entity of interest to be delivered into a target cell for the preparation of the conjugate of claim 1.

14. A method of preparing a PAR agonist or a conjugate capable of triggering target specific cellular uptake of an entity of interest comprising:
(a) coupling a spacer molecule (3) at the free amine group of the N-terminal serine of a PAR ligand, preferably a PAR activating peptide (4) so as to obtain an intermediate modified PAR ligand (5, 6), preferably a PAR agonist (6); and optionally
(b) coupling a bi-or multivalent linker (1, 2) which provides the ability to be chemically linked to the entity of interest to the spacer molecule of the modified PAR ligand (6), preferably wherein the linker comprises a detectable label (7), so as to obtain a further modified PAR ligand (8); and optionally
(c) conjugating the entity of interest to the linker, preferably via a free amino group of the linker so as to obtain a conjugate of the PAR ligand and the entity of interest.

15. The method of claim 14, wherein prior, during or after any one of steps (a) to (c) the potential intermolecular receptor-ligand interaction is determined based on ligand-docking simulations using a homology model of PAR, preferably human PAR2 together with the intermediate ligand or conjugate either with or without the entity of interest.


SEQUENCE LISTING (3 sequences)

SEQ ID NO: 1 - protein, 6 aa, Artificial Sequence
  PAR2 agonist peptide derived from rodent PAR2 TL
  PEPTIDE (1)..(6): PAR2 agonist peptide derived from rodent PAR2 TL, wherein the leucin at position 6 of the peptide is either modified with an NH2 group or remains unmodified
SLIGRL

SEQ ID NO: 2 - protein, 5 aa, Artificial Sequence
  Derivative of the PAR2 agonist peptide derived from rodent PAR2 TL
  PEPTIDE (1)..(5): Derivative of the PAR2 agonist peptide derived from rodent PAR2 TL, wherein the leucin at position 5 of the peptide is either modified with an NH2 group or remains unmodified and wherein the leucin at position 1 of the peptide is modified with a 2-furoyl group
LIGRL

SEQ ID NO: 3 - protein, 6 aa, Artificial Sequence
  PAR2 agonist peptide derived from human PAR2 TL
  PEPTIDE (1)..(6): PAR2 agonist peptide derived from human PAR2 TL, wherein the valine at position 6 of the peptide is either modified with an NH2 group or remains unmodified
SLIGKV